(19) United States
(12) Patent Application Publication
(10) Pub. No.: US 2009/0286294 A1
(43) Pub. Date: Nov. 19, 2009

(54) Methods and Compositions for Improving the Production of Fuels in Microorganisms

(75) Inventors: JEFFREY BLANCHARD, LEVERETT, MA (US); SUSAN LESCHINE, LEVERETT, MA (US); ELSA PETIT, NORTHAMPTON, MA (US); JOHN FABEL, AMHERST, MA (US)

Correspondence Address: FISH & RICHARDSON PC, P.O. BOX 1022, MINNEAPOLIS, MN, 55440-1022, US

(73) Assignee: UNIVERSITY OF MASSACHUSETTS, BOSTON, MA (US)

(21) Appl. No.: 12/419,211

(22) Filed: Apr. 6, 2009

Related U.S. Application Data
(60) Provisional application No. 61/042,657, filed on Apr. 4, 2008.

(51) Int. Cl. C12P 7/06; C12N 15/63; C12N 1/00; C12N 1/21; C12N 1/19

(52) U.S. Cl. 435/161; 536/23.2; 435/320.1; 435/243; 435/252.33; 435/252.3; 435/254.21; 435/254.22; 435/254.23; 435/252.34; 435/252.31

(57) ABSTRACT

The invention relates to compositions, systems, and methods for producing fuels, such as ethanol and hydrogen, and related compounds. More specifically, compositions and methods are provided for making recombinant microorganisms for the production of fuels using genes from the Clostridium phytofermentans ethanol and hydrogen pathways disclosed herein.

### CROSS REFERENCE TO RELATED APPLICATION

This application claims the benefit of priority from U.S. Provisional Patent Application No. 61/042,657, filed on Apr. 4, 2008, the contents of which is incorporated herein by reference in its entirety.

### BACKGROUND OF THE INVENTION

Compositions and methods are disclosed for engineering microorganisms that are capable of producing a fuel when grown in a variety of fermentation conditions. In certain embodiments, the methods comprise genetically engineering a microorganism to direct fuel production via the Clostridium phytofermentans ethanol pathway.

There is an interest in developing methods of producing usable energy from renewable and sustainable biomass resources. Energy in the form of carbohydrates can be found in waste biomass, and in dedicated energy crops, such as grains (e.g., corn or wheat) or grasses (e.g., switchgrass). Cellulosic and lignocellulosic materials, are produced, processed, and used in large quantities in a number of applications.

A current challenge is to develop viable and economical strategies for the conversion of carbohydrates into usable energy forms. Strategies for deriving useful energy from carbohydrates include the production of ethanol (“cellulosic ethanol”) and other alcohols (e.g., butanol), conversion of carbohydrates into hydrogen, and direct conversion of carbohydrates into electrical energy through fuel cells. For example, biomass ethanol strategies are described by DiPardo, Journal of Outlook for Biomass Ethanol Production and Demand (EIA Forecasts), 2002; Sheehan, Biotechnology Progress, 15:8179, 1999; Martin, Enzyme Microbes Technology, 31:274, 2002; Greer, BioCycle, 61-65, April 2005; Lynd, Microbiology and Molecular Biology Reviews, 66:3, 506-577, 2002; and Lynd et al. in “Consolidated Bioprocessing of Cellulosic Biomass: An Update,” Current Opinion in Biotechnology, 16:577-583, 2005.

### SUMMARY

The present disclosure relates to specific new isolated nucleic acid molecules that correspond to genes found in Clostridium phytofermentans (“C. phy”) that we have discovered are involved in C. phy's ability to produce various fuels from a wide variety of biomass materials. These new isolated nucleic acid molecules can be used to prepare expression vectors, which, in turn, can be used to engineer new recombinant microorganisms that can express these nucleic acid molecules to produce fuels. Certain polynucleotides, expression cassettes, expression vectors, and recombinant microorganisms for the optimization of ethanol production are disclosed in accordance with various embodiments of the present invention, as well as methods for making recombinant microorganisms that are capable of producing one or more fuels when grown under a variety of fermentation conditions.

In one aspect, the invention features isolated polynucleotides that encodes one or more polypeptides that modulate fuel production in C. phytofermentans. For example, polynucleotide can include a nicotinamide adenine dinucleotide (NADH) ferredoxin oxidoreductase (Nfo) subunit as described herein. The polynucleotide can include a C. phytofermentans rnf operon, e.g., a nucleic acid sequence corresponding to a region of the C. phytofermentans chromosome extending from about position 259945 to about position 265175.

In some embodiments the polynucleotide includes at least one nucleic acid sequence selected from the group consisting of SEQ ID NO: 1, SEQ ID NO:2, SEQ ID NO:3, SEQ ID NO:4, SEQ ID NO:5, and SEQ ID NO:6, and the Nfo subunit can be selected from the group consisting of RnfC, RnfD, RnfG, RnfE, RnfA, and RnfB. In certain embodiments the polynucleotide includes a nucleic acid sequence encoding any one or more, e.g., all, of subunits RnfC, RnfD, RnfG, RnfE, RnfA, and RnfB.

In certain embodiments, the polynucleotide can further include a nucleic acid sequence encoding an enzyme selected from the group consisting of pyruvate ferredoxin oxidoreductase (Pfo), acetaldehyde dehydrogenase, ethanol dehydrogenase, and hydrogenase.

In another aspect, the invention features expression cassettes (and vectors) that enable an organism to produce a fuel, the expression cassettes including an isolated polynucleotide that encodes at least one polypeptide that modulates fuel production in C. phytofermentans.

In some embodiments, the expression cassette includes a polynucleotide including a nucleic acid sequence encoding an Nfo subunit. In some embodiments, the expression cassettes include the C. phy rnf operon. In certain embodiments, the expression cassettes can further include a promoter. In some embodiments, the polynucleotides can further include a nucleic acid sequence encoding any one or more of pyruvate ferredoxin oxidoreductase (Pfo), an acetaldehyde dehydrogenase, an ethanol dehydrogenase, or a hydrogenase.

The invention also features recombinant microorganisms for producing one or more fuels. In some embodiments, the recombinant microorganisms include one or more polynucleotides that each includes a nucleic acid sequence encoding an Nfo subunit. In some embodiments, the polynucleotides include the C. phy rnf operon. In some embodiments, the recombinant microorganisms further include nucleic acid sequence encoding an enzyme selected from the group consisting of a Pfo, an acetaldehyde dehydrogenase, an ethanol dehydrogenase, and a hydrogenase. In some embodiments, the recombinant microorganism can be a cellulolytic or saccharolytic microorganism.

In some embodiments, the microorganism can be Clostridium cellulovorans, Clostridium cellulolyticum, Clostridium thermocellum, Clostridium josui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Acetivibrio cellulolyticus, Bacteroides cellulosolvens, Caldicellulosiruptor saccharolyticum, Ruminococcus albus, Ruminococcusflavefaciens, Fibrobacter succinogenes, Eubacterium cellulosolvens, Butyrivibrio fibrisolvens, Anaerocellum thermophilum, Halocella cellulolytica, Thermoanaerobacterium thermosaccharolyticum or Thermoanaerobacterium saccharolyticum. In some embodiments, the recombinant microorganism is capable of producing ethanol in recoverable quantities greater than about 10 mM ethanol after a 5 day fermentation.

In another aspect, the invention features methods of producing ethanol and other fuels, such as hydrogen. In certain of these embodiments, the methods include culturing one or more different recombinant microorganisms in a culture medium, wherein the recombinant microorganisms include a nucleic acid sequence encoding an Nfo subunit; and accumulating ethanol in the culture medium. In some embodiments, the recombinant microorganism includes the C. phy rnf operon. In some embodiments, the recombinant microorganism includes an expression cassette including a nucleic acid sequence encoding an Nfo subunit. In some embodiments, the recombinant microorganism is capable of expressing Nfo.

As utilized in accordance with the embodiments provided herein, the following terms, unless otherwise indicated, shall be understood to have the following meanings:

“Nucleotide” refers to a phosphate ester of a nucleoside, as a monomer unit or within a nucleic acid. “Nucleotide 5′-triphosphate” refers to a nucleotide with a triphosphate ester group at the 5′ position, and are sometimes denoted as “NTP”, or “dNTP” and “ddNTP” to particularly point out the structural features of the ribose sugar. The triphosphate ester group can include sulfur substitutions for the various oxygens, e.g., α-thio-nucleotide 5′-triphosphates. For a review of nucleic acid chemistry, see: Shabarova, Z. and Bogdanov, A. Advanced Organic Chemistry of Nucleic Acids, VCH, New York, 1994.

The terms “nucleic acid” and “nucleic acid molecule” refer to natural nucleic acid sequences, artificial nucleic acids, analogs thereof, or combinations thereof.

The terms “polynucleotide” and “oligonucleotide” are used interchangeably and mean single-stranded and double-stranded polymers of nucleotide monomers (nucleic acids), including, but not limited to, 2′-deoxyribonucleotides (DNA) and ribonucleotides (RNA) linked by internucleotide phosphodiester bond linkages, e.g., 3′-5′ and 2′-5′, inverted linkages, e.g., 3′-3′ and 5′-5′, branched structures, or analog nucleic acids. Polynucleotides have associated counter ions, such as H⁺, NH₄⁺, trialkylammonium, Mg₂⁺, Na⁺, and the like. A polynucleotide can be composed entirely of deoxyribonucleotides, entirely of ribonucleotides, or chimeric mixtures thereof. Polynucleotides can be comprised of nucleobase and sugar analogs. Polynucleotides typically range in size from a few monomeric units, e.g., 5-40, when they are more commonly frequently referred to in the art as oligonucleotides, to several thousands of monomeric nucleotide units. Unless denoted otherwise, whenever a polynucleotide sequence is represented, it will be understood that the nucleotides are in 5′ to 3′ order from left to right, and that “A” denotes deoxyadenosine, “C” denotes deoxycytidine, “G” denotes deoxyguanosine, and “T” denotes thymidine.

A polypeptide or protein that “modulates” a particular biological process is a polypeptide that is involved in the positive or negative regulation of that process, e.g., to enhance or to inhibit that process. For example, as disclosed herein there are many proteins that modulate, e.g., enable, enhance, or increase, fuel production in C. phytofermentans. Thus, as referred to herein “an isolated polynucleotide that encodes at least one polypeptide that modulates fuel production in C. phytofermentans” means that the polynucleotide comprises a sequence of nucleotides that is the same as a corresponding sequence present in C. phy that is disclosed herein as regulating fuel production. This phrase does not require that the sequence be physically removed from C. phy, only that the sequence is the same. For example, the sequence may have been generated synthetically. Of course, variants (e.g., mutant forms) as described herein are also contemplated, such as variant nucleic acid sequences that encode the same or similar polynucleotide, or variant polynucleotide sequences that have the same or essentially the same biological activity as the C. phy sequences recited herein.

The term “fuel” is used herein to refer to compounds suitable as liquid or gaseous fuels including, but not limited to, hydrocarbons, hydrogen, methane, and hydroxy compounds such as alcohols (e.g., ethanol, butanol, propanol, methanol, and mixtures thereof). The term “chemicals” is used herein to refer to carbonyl compounds such as aldehydes and ketones (e.g., acetone, formaldehyde, and 1-propanal), organic acids, derivatives of organic acids such as esters (e.g., wax esters and glycerides), and other functional compounds including, but not limited to, 1,2-propanediol, 1,3-propanediol, lactic acid, formic acid, acetic acid, succinic acid, pyruvic acid, enzymes such as cellulases, polysaccharases, lipases, proteases, ligninases, and hemicellulases.

The terms “nicotinamide adenine dinucleotide ferredoxin oxidoreductase,” “NADH ferredoxin oxidoreductase,” and “Nfo” are used interchangeably and refer to an enzyme that catalyzes the chemical reaction: reduced ferredoxin+NAD⁺⇄oxidized ferredoxin+NADH+H⁺.

The term “plasmid” refers to a circular nucleic acid vector. Generally, plasmids contain an origin of replication that allows many copies of the plasmid to be produced in a bacterial (or sometimes eukaryotic) cell without integration of the plasmid into the host cell DNA.

The term “construct” as used herein refers to a recombinant nucleotide sequence, generally a recombinant nucleic acid molecule, that has been generated for the purpose of the expression of a specific nucleotide sequence(s), or is to be used in the construction of other recombinant nucleotide sequences. In general, “construct” is used herein to refer to a recombinant nucleic acid molecule.

An “expression cassette” refers to a set of polynucleotide elements that permit transcription of a polynucleotide in a host cell. Typically, the expression cassette includes a promoter and a heterologous or native polynucleotide sequence that is transcribed. Expression cassettes may also include additional nucleic acid sequences, e.g., transcription termination signals, polyadenylation signals, and enhancer elements.

By “expression vector” is meant a vector that permits the expression of a polynucleotide, e.g., one or more expression cassettes, inside a cell. Expression of a polynucleotide includes transcriptional and/or post-transcriptional events. An “expression construct” is an expression vector into which a nucleotide sequence of interest has been inserted in a manner so as to be positioned to be operably linked to the expression sequences present in the expression vector.

An “operon” refers to a set of polynucleotide elements that produce a messenger RNA (mRNA). Typically, the operon includes a promoter and one or more structural genes. Typically, an operon contains one or more structural genes which are transcribed into one polycistronic mRNA: a single mRNA molecule that codes for more than one protein. In some embodiments, an operon may also include an operator which regulates the activity of the structural genes of the operon.

The term “host cell” refers to a cell that is to be transformed using the methods and compositions of the invention. In general, host cell as used herein means a microorganism cell into which a nucleic acid of interest is to be transformed.

The term “transformation” refers to a permanent or transient genetic change, preferably a permanent genetic change, induced in a cell following incorporation of non-host nucleic acid sequences.

The term “transformed cell” refers to a cell into which (or into an ancestor of which) has been introduced, by means of recombinant nucleic acid techniques, a nucleic acid molecule encoding a gene product (e.g., RNA and/or protein) of interest (e.g., nucleic acid encoding a cellular product).

The term “gene” refers to any and all discrete coding regions of a host genome, or regions that code for a functional RNA only (e.g., tRNA, rRNA, and regulatory RNAs such as ribozymes). Genes can thus include associated non-coding regions and optionally regulatory regions, as well as open reading frames encoding specific polypeptides, introns, and adjacent 5′ and 3′ non-coding nucleotide sequences involved in the regulation of expression. A gene may further include control signals such as promoters, enhancers, termination and/or polyadenylation signals that are naturally associated with a given gene, or heterologous control signals. The gene sequences may be cDNA or genomic nucleic acid or a fragment thereof. The gene may be introduced into an appropriate vector for extrachromosomal maintenance or for integration into the host.

The terms “gene of interest,” “nucleotide sequence of interest” “polynucleotide of interest” or “nucleic acid of interest” refer to any nucleotide or nucleic acid sequence that encodes a protein or other molecule that is desirable for expression in a host cell (e.g., for production of the protein or other biological molecule (e.g., an RNA product) in the target cell). The nucleotide sequence of interest is generally operatively linked to other sequences which are needed for its expression, e.g., a promoter.

The term “promoter” refers to a minimal nucleic acid sequence sufficient to direct transcription of a nucleic acid sequence to which it is operably linked. The term “promoter” is also meant to encompass those promoter elements sufficient for promoter-dependent gene expression controllable for cell-type specific expression, tissue-specific expression, or inducible by external signals or agents; such elements may be located in the 5′ or 3′ regions of the naturally-occurring gene. The term “inducible promoter” refers to a promoter that is transcriptionally active when bound to a transcriptional activator, which in turn is activated under a specific condition(s), e.g., in the presence of a particular chemical signal or combination of chemical signals that affect binding of the transcriptional activator, e.g., CO₂ or NO₂, to the inducible promoter and/or affect function of the transcriptional activator itself.

The terms “operator,” “control sequence,” or “regulatory sequence” refer to nucleic acid sequences that regulate the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

By “operably connected” or “operably linked” and the like is meant a linkage of polynucleotide elements in a functional relationship. A nucleic acid sequence is “operably linked” when it is placed into a functional relationship with another nucleic acid sequence. For instance, a promoter or enhancer is operably linked to a coding sequence if it affects the transcription of the coding sequence. In some embodiments, operably linked means that the nucleic acid sequences being linked are typically contiguous and, where necessary to join two protein coding regions, contiguous and in reading frame. A coding sequence is “operably linked” to another coding sequence when RNA polymerase will transcribe the two coding sequences into a single mRNA, which is then translated into a single polypeptide having amino acids derived from both coding sequences. The coding sequences need not be contiguous to one another so long as the expressed sequences are ultimately processed to produce the desired protein.

“Operably connecting” a promoter to a transcribable polynucleotide means placing the transcribable polynucleotide (e.g., protein encoding polynucleotide or other transcript) under the regulatory control of a promoter, which then controls the transcription, and optionally translation, of that polynucleotide. In the construction of heterologous promoter/structural gene combinations, it is generally preferred to position a promoter or variant thereof at a distance from the transcription start site of the transcribable polynucleotide, which is approximately the same as the distance between that promoter and the gene it controls in its natural setting, i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting, i.e., the genes from which it is derived.

The term “derived” means that a specific gene, nucleic acid sequence, or amino acid sequence, is either obtained directly (e.g., by physical manipulation) from a specific source, such as a naturally occurring gene or protein, e.g., a wild type sequence, or is prepared, e.g., synthetically, to have the same or similar sequence as that of a portion of the specific source.

“Culturing” signifies incubating a cell or organism under conditions wherein the cell or organism can carry out some, if not all, biological processes. For example, a cell that is cultured may be growing or reproducing, or it may be non-viable, but still capable of carrying out biological and/or biochemical processes such as replication, transcription, translation, etc.

By “transgenic organism” is meant a non-human organism, e.g., a single-cell organism (e.g., a microorganism), a mammal (e.g., a laboratory, domesticated, or farm animal), or a non-mammal (e.g., a fish, worm (e.g., a nematode), or insect (e.g., a Drosophila)), having a non-endogenous (i.e., heterologous) nucleic acid sequence present in at least some of its cells or stably integrated into its germ line nucleic acid.

The term “biomass,” as used herein refers to a mass of living or biological carbon-containing materials and includes natural, processed, organic, and/or synthetic materials. The various types of biomass include plant biomass and municipal waste biomass (residential and light commercial refuse with recyclables such as metal and glass removed). The terms “plant biomass” and “lignocellulosic biomass” refer to any plant-derived organic matter (woody or non-woody) available for energy on a sustainable or renewable basis. Examples of biomass include paper, paper products, paper waste, wood, particle board, sawdust, agricultural waste, sewage, silage, grasses, rice hulls, bagasse, cotton, jute, hemp, flax, bamboo, sisal, abaca, straw, corn cobs, corn stover, switchgrass, alfalfa, hay, rice hulls, coconut hair, cotton, synthetic celluloses, seaweed, algae, or mixtures of these.

“Recombinant polynucleotides” are polynucleotides synthesized or otherwise manipulated in vitro. Recombinant polynucleotides can be used to produce gene products encoded by those polynucleotides in cells or other biological systems. For example, a cloned polynucleotide may be inserted into a suitable expression vector, such as a bacterial plasmid, and the plasmid can be used to transform a suitable host cell. A host cell that comprises the recombinant polynucleotide is referred to as a “recombinant host cell” or a “recombinant bacterium.” The gene is then expressed in the recombinant host cell to produce, e.g., a “recombinant protein.” A recombinant polynucleotide may serve a non-coding function (e.g., promoter, origin of replication, ribosome-binding site, etc.) as well.

“Biocatalysts” are enzymes and/or microorganisms that serve to induce or enhance a particular reaction. In some contexts this word refers to the possible use of either enzymes or microorganisms to serve a particular function, in other contexts the word will refer to the combined use of the two, and in other contexts the word will refer to only one of the two. The context of the phrase will indicate the meaning intended to one of skill in the art.

The term “homologous” recombination refers to the process of recombination between two nucleic acid molecules based on nucleic acid sequence similarity. The term embraces both reciprocal and nonreciprocal recombination (also referred to as gene conversion). In addition, the recombination can be the result of equivalent or non-equivalent cross-over events. Equivalent crossing over occurs between two equivalent sequences or chromosome regions, whereas nonequivalent crossing over occurs between identical (or substantially identical) segments of nonequivalent sequences or chromosome regions. Unequal crossing over typically results in gene duplications and deletions. For a description of the enzymes and mechanisms involved in homologous recombination see, Watson et al., Molecular Biology of the Gene pp 313-327, The Benjamin/Cummings Publishing Co. 4th ed. (1987).

The terms “non-homologous” or “random” integration refer to any process by which nucleic acid is integrated into a genome in a manner that does not involve homologous recombination. It appears to be a arbitrary process in which incorporation can occur at any of a large number of genomic locations.

A “heterologous polynucleotide” or a “heterologous nucleic acid” is a polynucleotide that is functionally related to another polynucleotide, such as a promoter sequence, in a manner so that the two polynucleotide sequences are not arranged in the same relationship to each other as in nature. Heterologous polynucleotide sequences include, e.g., a promoter operably linked to a heterologous nucleic acid, and a polynucleotide including its native promoter that is inserted into a heterologous vector for transformation into a recombinant host cell. Heterologous polynucleotide sequences are considered “exogenous,” because they are introduced into the host cell via transformation techniques. However, the heterologous polynucleotide can originate from a foreign cell or from the same type of cell. Modification of the heterologous polynucleotide sequence may occur, e.g., by treating the polynucleotide with a restriction enzyme to generate a polynucleotide sequence that can be operably linked to a regulatory element. Modification can also occur by techniques such as site-directed mutagenesis.

A polynucleotide that is “endogenously expressed” refers to a polynucleotide that is natively produced by a host cell without external manipulation or the insertion of a new genetic sequence.

A host cell that is “competent to express” a protein is a host cell that provides a sufficient cellular environment for expression of endogenous and/or exogenous polynucleotides.

All numbers expressing quantities of ingredients, reaction conditions, and so forth used in the specification and claims are to be understood as being modified in all instances by the term “about.” Accordingly, unless indicated to the contrary, the numerical parameters set forth in the specification and attached claims are approximations that can vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. When definitions of terms in incorporated references appear to differ from the definitions provided in the present teachings, the definition provided in the present disclosure shall control.

The section headings used herein are for organizational purposes only and are not to be construed as limiting the described subject matter in any way. In addition, the materials, methods, and examples are illustrative only and not intended to be limiting. The use of the singular includes the plural unless specifically stated otherwise. Also, the use of “comprise,” “comprises,” “comprising,” “contain,” “contains,” “containing,” “include,” “includes,” and “including” are not intended to be limiting. It is to be understood that both the foregoing general description and the following detailed description are exemplary and explanatory only and are not restrictive of the invention. The articles “a” and “an” are used herein to refer to one or to more than one (i.e., to at least one) of the grammatical object of the article. By way of example, “an element” means one element or more than one element.

Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000).

Other features and advantages of the invention will be apparent from the following detailed description, and from the claims.

### BRIEF DESCRIPTION OF THE DRAWINGS

FIG. 1 is a schematic diagram of the Clostridium phytofermentans ethanol pathway. The letters A-E represent the following enzymes: A, pyruvate ferredoxin oxidoreductase (Pfo); B, nicotinamide adenine dinucleotide (NADH) ferredoxin oxidoreductase (Nfo); C, acetaldehyde dehydrogenase; D ethanol dehydrogenase; and E hydrogenase.

FIGS. 2A to 2C are a series of three graphs illustrating the rank abundance of mRNA expression levels for rnfB determined from microarray experiments and plotted as a function of genome-wide mRNA ranking when C. phy is cultured on three exemplary carbon sources: glucose (FIG. 2A), cellulose (FIG. 2B), or xylan (FIG. 2 C). These results support a central role of the rnf genes in C. phy metabolism of cellulosic materials to produce fuels.

### DETAILED DESCRIPTION

The present disclosure relates to specific new isolated nucleic acid molecules that correspond to genes present in Clostridium phytofermentans that we have discovered are involved in C. phy's ability to produce various fuels such as ethanol and hydrogen from a wide variety of biomass materials. These new isolated nucleic acid molecules can thus be used to prepare expression vectors, which, in turn, can be used to engineer new recombinant microorganisms that can express these nucleic acid molecules to modulate fuel production by these microorganisms. Polynucleotides, expression cassettes, expression vectors, and recombinant microorganisms for the optimization of ethanol production are disclosed in accordance with various embodiments of the present invention.

Various embodiments disclosed herein are generally directed towards compositions and methods for making recombinant microorganisms that are capable of producing a fuel when grown under a variety of fermentation conditions and with a variety of carbon sources. Generally, a recombinant microorganism can efficiently and stably produce a fuel, such as ethanol or hydrogen, and related compounds, so that a high yield of fuel is provided from relatively inexpensive raw biomass materials such as, for example, cellulose.

At present, there are a limited number of techniques that exist for making recombinant organisms that are capable of producing a fuel. The various techniques often have problems that can lead to low fuel yield, high cost, and undesirable by-products. Until now, recombinant microorganism strategies have generally utilized pyruvate decarboxylase (pdc) and alcohol dehydrogenase (adh) to generate recombinant microorganisms that are capable of producing fuels. However, these strategies involve an energy loss in the host organism, because energy is not conserved. Some of the embodiments described herein overcome this and other limitations.

In some embodiments, polynucleotides and expression cassettes for an efficient fuel-producing system are provided. The polynucleotides and expression cassettes can be used to prepare expression vectors for transforming microorganisms to confer upon the transformed microorganisms the capability of producing fuel in useful quantities.

In some embodiments, the metabolism of a microorganism can be modified by introducing and expressing various genes. In accordance with some embodiments of the present invention, the recombinant microorganisms can use genes from Clostridium phytofermentans (ISDgT, American Type Culture Collection 700394T, referred to herein as “C. phy”) as a biocatalyst for the enhanced conversion of, for example, cellulose, to a fuel, such as ethanol and/or hydrogen. Various expression vectors can be introduced into a host microorganism so that the transformed microorganism can produce large quantities of fuel in various fermentation conditions. The recombinant microorganisms are preferably modified so that a fuel is stably produced with high yield when grown on a medium comprising, for example, cellulose.

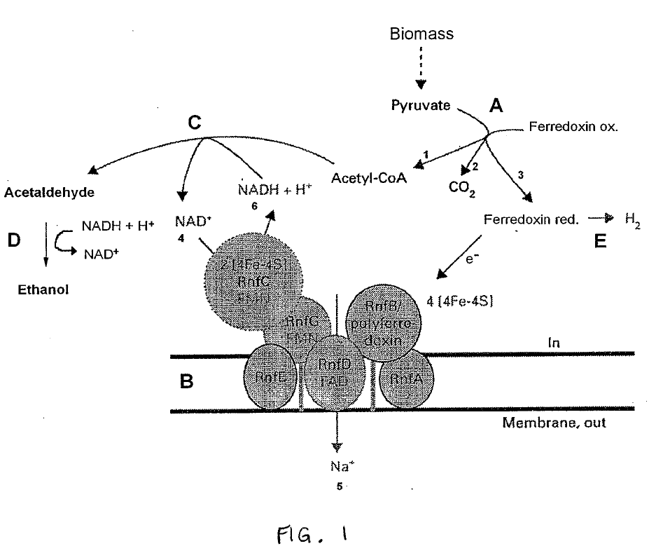

C. phy, alone or in combination with one or more other microbes, can ferment on a large scale a cellulosic biomass material into a combustible biofuel, such as, ethanol, propanol, and/or hydrogen (see, e.g., U.S. Patent Application No. 2007/0178569; Warnick et. al., Int J Syst Evol Microbiol (2002), 52 1155-1160, each of which is herein incorporated by reference in its entirety). It has been newly discovered that C. phy utilizes a pathway involving nicotinamide adenine dinucleotide (NADH) ferredoxin oxidoreductase (Nfo) for producing ethanol and hydrogen. FIG. 1 shows a schematic diagram of the C. phy ethanol pathway. In this pathway, the oxidative decarboxylation of pyruvate catalyzed by pyruvate ferredoxin oxidoreductase (Pfo) yields acetyl-CoA (1), carbon dioxide (2) and reduced ferredoxin (3) (FIG. 1 at A).

The reduced ferredoxin is reoxidized in two different pathways. One pathway involves Nfo to produce NADH. The other pathway uses hydrogenase to form hydrogen. In the Nfo pathway, Nfo catalyzes the reduction of NAD⁺ (4) by reduced ferredoxin (3) to generate an electrochemical Na⁺ gradient (5) (FIG. 1 at B). NADH (6) is generated as a product of this reaction. The NADH can serve as a substrate for acetaldehyde dehydrogenase, which catalyzes the reduction of acetyl-CoA to acetaldehyde (see, FIG. 1 at C). Acetaldehyde is then reduced to ethanol by ethanol dehydrogenase (FIG. 1 at D). In the hydrogenase pathway, hydrogen is produced when hydrogenase catalyzes the transfer of electrons from reduced ferredoxin to protons (see, FIG. 1 at E).

Nfo is a membrane-bound enzyme complex that uses the energy difference between reduced ferredoxin and NADH to generate an electrochemical Na⁺ gradient. The rnf operon of C. phy, which has been newly identified, encodes C. phy Nfo. The C. phy rnf operon includes at least six genes that encode subunits of Nfo. The genes of the C. phy rnf include: Cphy0211, Cphy0212, Cphy0213, Cphy0214, Cphy0215 and Cphy0216, which encode the Nfo subunits RnfC, RnfD, RnfG, RnfE, RnfA, and RnfB, respectively (see Table 1). Although Nfo was previously shown to be involved in other pathways, such as the 3-methylaspartate pathway in Clostridium tetanomorphum and the 2-hydroxyglutarate pathway in Acidaminococcus fermentans and Fusobacterium nucleatum (Boiangiu et al., J. Mol. Microbiol. Biotechnol. 10: 105-119, 2005), until now, Nfo's role in ethanol production was unknown.

The polynucleotides, expression cassettes, and expression vectors disclosed herein can be inserted into many different host microorganisms using standard techniques to provide these host organisms with the ability to produce one or more fuels such as ethanol and hydrogen. For example, in addition to C. phy, cellulolytic microorganisms such as Clostridium cellulovorans, Clostridium cellulolyticum, Clostridium thermocellum, Clostridiumjosui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Acetivibrio cellulolyticus, Bacteroides cellulosolvens, Caldicellulosiruptor saccharolyticum, Ruminococcus albus, Ruminococcusflavefaciens, Fibrobacter succinogenes, Eubacterium cellulosolvens, Butyrivibrio fibrisolvens, Anaerocellum thermophilum, and Halocella cellulolytica are particularly attractive hosts, because they are capable of hydrolyzing cellulose. Other microorganisms that can be used include, for example, Saccharolytic microbes such as Thermoanaerobacterium thermosaccharolyticum and Thermoanaerobacterium saccharolyticum. Additional potential hosts include other bacteria, yeasts, algae, fungi, and eukaryotic cells.

In various embodiments, the polynucleotides, expression cassettes, and expression vectors disclosed herein can be used with C. phy or other Clostridia to increase the production of fuel such as ethanol and hydrogen.

In some embodiments the polynucleotides include C. phy genes encoding the Nfo subunits together with appropriate regulatory sequences. The regulatory sequences may consist of promoters, inducers, operators, ribosomal binding sites, terminators, and/or other regulatory sequences. Fuel production in previous recombinant systems was dependent upon native activities in the host organisms. Advantageously, the dependence upon endogenous host genes is now eliminated by providing C. phy genes encoding Nfo subunits. In some embodiments, expression cassettes are provided that include a gene encoding another enzyme involved in the C. phy ethanol pathway, such as, for example, Pfo, acetaldehyde dehydrogenase, and ethanol dehydrogenase. In other embodiments, the expression cassettes can include a gene encoding a hydrogenase. For the C. phy ethanol pathway described herein, it is not necessary that the genes encoding each enzyme be under common control; they can be under separate control and even in different plasmids, or places on the chromosome.

As will be appreciated by one of skill in this field, the ability to produce recombinant organisms that can produce fuels can have great benefit, especially for efficient, cost-effective, and environmentally friendly fuel production.

Polynucleotides and Expression Cassettes

Some of the presently disclosed embodiments are directed to polynucleotides useful for the production of a fuel in a recombinant microorganism. Other embodiments are directed to expression cassettes for expression of one or more polynucleotides of interest for the production of a fuel in a recombinant microorganism. In certain embodiments, a polynucleotide comprising the C. phy rnf operon is provided. In some embodiments, a polynucleotide sequence encoding each of the Nfo subunits RnfC, RnfD, RnfG, RnfE, RnfA, and RnfB is provided. In some embodiments, a polynucleotide of interest comprises the sequences of any one or more, or all of Cphy0211, Cphy0212, Cphy0213, Cphy0214, Cphy0215, and Cphy0216. These genes encode the C. phy Nfo subunits RnfC, RnfD, RnfG, RnfE, RnfA, and RnfB, respectively. The GenBank ID, locus and chromosome position information for various C. phy genes are provided in Table 1 below.

TABLE 1

| | | | Chromosome | | |
|---|---|---|---|---|---|
| Product Name | GenBank ID | Locus | Position | SEQ ID NO: | |
| NADH: ferredoxin | 160878369 | Cphy0211 | 259945 . . . 261264 | 1 | (amino acid) |
| oxidoreductase, subunit RnfC | | | | 10 | (nucleic acid) |
| NADH: ferredoxin | 160878370 | Cphy0212 | 261309 . . . 262319 | 2 | (amino acid) |
| oxidoreductase, subunit RnfD | | | | 11 | (nucleic acid) |
| NADH: ferredoxin | 160878371 | Cphy0213 | 262309 . . . 262965 | 3 | (amino acid) |
| oxidoreductase, subunit RnfG | | | | 12 | (nucleic acid) |
| NADH: ferredoxin | 160878372 | Cphy0214 | 262958 . . . 263719 | 4 | (amino acid) |
| oxidoreductase, subunit RnfE | | | | 13 | (nucleic acid) |
| NADH: ferredoxin | 160878373 | Cphy0215 | 263734 . . . 264309 | 5 | (amino acid) |
| oxidoreductase, subunit RnfA | | | | 14 | (nucleic acid) |
| NADH: ferredoxin | 160878374 | Cphy0216 | 264327 . . . 265175 | 6 | (amino acid) |
| oxidoreductase, subunit RnfB | | | | 15 | (nucleic acid) |
| Alcohol dehydrogenase | 160879180 | Cphy1029 | 1301846 . . . 1303036 | 7 | (amino acid) |
| | | | | 16 | (nucleic acid) |
| Acetaldehyde dehydrogenase | 160882043 | Cphy3925 | 4821675 . . . 4824293 | 8 | (amino acid) |
| | | | | 17 | (nucleic acid) |
| Pyruvate: ferredoxin | 160881678 | Cphy3558 | 4391888 . . . 4395415 | 9 | (amino acid) |
| oxidoreductase | | | | 18 | (nucleic acid) |

In some embodiments, the expression cassette comprises the whole rnf operon. The rnf operon can be, for example, the C. phy rnf operon. In some embodiments, the expression cassette comprises a polynucleotide having a sequence from the C. phy chromosome region spanning from about position 259345 to about position 265175. In some embodiments, the expression cassette comprises a polynucleotide having a sequence from the C. phy chromosome region spanning from about position 259945 to about position 265175. In some embodiments, the expression cassette comprises a polynucleotide sequence which is at least about 80, 85, 90, 95, 99, or about 100% identical to a sequence from the C. phy chromosome region spanning from about position 259945 to about position 265175. In some embodiments, the expression cassette comprises a polynucleotide having a sequence from at least a portion of the C. phy chromosome sequence from up to about 600 bases upstream of the start codon of Cphy0211 to the start codon of Cphy0261.

In some embodiments, a polynucleotide sequence encoding a subunit of Nfo is provided. In certain embodiments, the polynucleotide sequence encodes all of the Nfo subunits. Any polynucleotide sequence encoding an Nfo subunit, e.g., RnfC, RnfD, RnfG, RnfE, RnfA, and RnfB, which is capable of being expressed, can be used in the present invention. In some embodiments, a polynucleotide sequence encoding an Nfo subunit can be a C. phy Nfo subunit gene. In certain embodiments, the genes encoding the Nfo subunits include Cphy0211, Cphy0212, Cphy0213, Cphy0214, Cphy0215, and Cphy0216, which encode the C. phy Nfo subunits RnfC, RnfD, RnfG, RnfE, RnfA and RnfB, respectively. In some embodiments, an expression cassette comprises a polynucleotide having a sequence at least about 80, 85, 90, 95, 99, or 100% identical to a sequence encoding a C. phy Nfo subunit.

If the polynucleotide or polypeptide is not 100% identical to the corresponding C. phy polynucleotide or polypeptide disclosed herein, it is referred to herein as a variant polynucleotide or poly peptide. For example, a variant polynucleotide can encode the identical polypeptide as a polynucleotide that is 100% identical to the C. phy sequences disclosed herein. Similarly, a variant polypeptide may have the same or essentially the same biological function as a polypeptide disclosed herein. A variant polypeptide may have at least 50, 60, 70, 75, 80, 85, 90, 95, 98, or 99% of the biological function, e.g., modulation of ethanol or hydrogen production, as a wild type C. phy polypeptide disclosed herein. Some variant polypeptides can have even greater than 100% of the wild type function.

In some embodiments, a sequence encoding a C. phy Nfo subunit comprises the sequence of the C. phy chromosome regions shown in Table 1 above.

In some embodiments, an expression cassette comprises a polynucleotide encoding one or more of the following amino acid sequences: SEQ ID NO:1 (RnfC), SEQ ID NO:2 (RnfD), SEQ ID NO:3 (RnfG), SEQ ID NO:4 (RnfE), SEQ ID NO:5 (RnfA) and SEQ ID NO:6 (RnfB). In some embodiments, an expression cassette comprises a polynucleotide encoding the amino acid sequences of SEQ ID NO:1, SEQ ID NO:2, SEQ ID NO:3, SEQ ID NO:4, SEQ ID NO:5 and SEQ ID NO:6. In some embodiments, an expression cassette comprises a polynucleotide comprising one or more of the following nucleic acid sequences: SEQ ID NO: 10 (RnfC), SEQ ID NO: 11 (RnfD), SEQ ID NO: 12 (RnfG), SEQ ID NO: 13 (RnfE), SEQ ID NO:14 (RnfA), and SEQ ID NO:15 (RnfB).

In other embodiments, an expression cassette comprising a polynucleotide sequence encoding Pfo, acetaldehyde dehydrogenase, alcohol or ethanol dehydrogenase, hydrogenase, or a combination thereof, is provided. In some embodiments, the polynucleotide encoding alcohol dehydrogenase comprises the sequence of Cphy1029. In some embodiments, an expression cassette comprises a polynucleotide encoding the amino acid sequence of SEQ ID NO:7 (C phy alcohol dehydrogenase). In some embodiments, the polynucleotide encoding alcohol dehydrogenase comprises the nucleic acid sequence of SEQ ID NO: 16. In some embodiments, the polynucleotide encoding acetaldehyde dehydrogenase comprises the sequence of Cphy3925. In some embodiments, an expression cassette comprises a polynucleotide encoding the amino acid sequence of SEQ ID NO:8 (C phy acetaldehyde dehydrogenase). In some embodiments, the polynucleotide encoding alcohol dehydrogenase comprises the nucleic acid sequence of SEQ ID NO: 17. In some embodiments, the polynucleotide encoding Pfo comprises the sequence of Cphy3558. In some embodiments, an expression cassette comprises a polynucleotide encoding the amino acid sequence of SEQ ID NO:9 (C phy Pfo). In some embodiments, the polynucleotide encoding Pfo comprises the nucleic acid sequence of SEQ ID NO: 18.

In some embodiments, the expression cassette comprises, or additionally comprises, a polynucleotide sequence(s) corresponding to any one or more of the following genes Cphy0086, Cphy0087, Cphy0088, Cphy0089, Cphy0090, Cphy0091, Cphy0092, and Cphy0093. These genes encode C Phy hydrogenase subunits. For example, the genes Cphy0087 (NCBI-GI: 160878248, chromosome position 115437 . . . 117140), Cphy0090 (NCBI-GI: 160878251, position 120033 . . . 121487), and Cphy0092 (NCBI-GI: 160878253, position 122755.124488) are subunits that we have found modulate hydrogen production. The nucleotide and corresponding amino acid sequences for these genes are available on various databases and the full sequences are incorporated herein by reference. The sequences of the other C. phy genes noted herein are similarly available on various databases under the Cphy gene numbers used herein.

In some embodiments, an expression cassette comprises at least a polynucleotide sequence encoding Nfo and a polynucleotide sequence encoding Pfo. In some embodiments, the expression cassette can further comprise a polynucleotide sequence encoding acetaldehyde dehydrogenase. In some embodiments, the expression cassette can further comprise a polynucleotide sequence encoding ethanol dehydrogenase.

In an expression cassette, the polynucleotide(s) of interest is operably linked to a promoter. Promoters suitable for the present invention include any promoter for expression of the polynucleotide of interest. In some embodiments, the promoter can be the natural promoter of the C. phy rnf operon. In some embodiments, the promoter can be an inducible promoter, such as, for example, a light-inducible promoter or a temperature sensitive promoter. In other embodiments, the promoter can be a constitutive promoter. In some embodiments, a promoter can be selected based upon the desired expression level for the polynucleotide(s) of interest in the host microorganism. In some embodiments, the promoter can comprise a polynucleotide having a sequence anywhere from at least a portion of the C. phy chromosome sequence from about 600 bases upstream of the start codon of Cphy0211 to the start codon of Cphy0261.

A typical expression cassette contains a promoter operably linked to one or more polynucleotides of interest. In some embodiments, the promoter can be positioned about the same distance from the heterologous transcription start site as it is from the transcription start site in its natural setting. As is known in the art, however, some variation in this distance can be accommodated without loss of promoter function. In some embodiments, a polynucleotide sequence comprising two or more genes encoding an Nfo subunit can have non-coding sequence between the coding sequences. In some embodiments, the expression cassette comprises the rnf operon of C. phy.

In certain embodiments, the polynucleotide sequences coding for each subunit of Nfo are under common control in an expression cassette. For example, the polynucleotide sequences coding for each subunit of Nfo are preferably operably linked to the same promoter. In some embodiments, all of the Nfo subunit genes can be transcribed into one polycistronic mRNA.

Standard molecular biology techniques known to those skilled in the art of recombinant nucleic acid and cloning can be applied to carry out the methods described herein unless otherwise specified. For example, the various fragments comprising the various constructs, expression cassettes, markers, and the like may be introduced by restriction enzyme cleavage of an appropriate replication system, and insertion of the particular construct or fragment into the available site. After ligation and cloning, the vector may be isolated for further manipulation. All of these techniques are amply explained in the literature and find exemplification in Maniatis et al., Molecular cloning: a laboratory manual, 3^{rd} ed. (2001) Cold Spring Harbor Laboratory Press, Cold Spring Harbor, N.Y.

In developing the constructs, the various polynucleotide fragments comprising the regulatory regions and open reading frame may be subjected to different processing conditions, such as ligation, restriction enzyme digestion, PCR, in vitro mutagenesis, linkers, and the like. Thus, nucleotide transitions, transversions, insertions, deletions, or the like, may be performed on the nucleic acid molecules employed in the regulatory regions or the nucleic acid sequences of interest for expression in the host microorganisms. Methods for restriction digests, Klenow blunt end treatments, ligations, and the like are well known to those in the art and are described, for example, by Maniatis et al. (2001).

During the preparation of the constructs, the various fragments of nucleic acid can be cloned in an appropriate cloning vector, which allows for amplification of the nucleic acid, modification of the nucleic acid or manipulation of the nucleic acid by joining or removing sequences, linkers, or the like. In some embodiments, the vectors will be capable of replication to at least a relatively high copy number in, for example, E. coli. A number of vectors are readily available for cloning, including such vectors as, for example, pBR322, vectors of the pUC series, the M13 series vectors, and pBluescript vectors (Stratagene; La Jolla, Calif.).

Expression Vectors

Expression vectors typically include one or more expression cassettes that contain all the elements required for the expression of one or more nucleic acids of interest in a host cell for the production of a fuel in a recombinant microorganism. In some embodiments, a polynucleotide of interest is introduced into a vector to create a recombinant expression vector suitable for transformation of a host cell for the production of a fuel in a recombinant microorganism. In other embodiments, an expression cassette can be introduced into a vector to create a recombinant expression vector suitable for transformation of a host cell. An expression vector can comprise an expression cassette comprising an rnf operon and another expression cassette comprising a polynucleotide encoding Pfo, acetaldehyde dehydrogenase, ethanol dehydrogenase, hydrogenase, or a combination thereof.

Expression vectors can replicate autonomously, or they can replicate by being inserted into the genome of the host cell, e.g., homologously or non-homogeneously integrated into the host cell genome. In some embodiments, the expression cassette can integrate into a desired locus via double homologous recombination.

In some embodiments, it can desirable for a vector to be usable in more than one host cell, e.g., in E. coli for cloning and construction, and in, e.g., a Clostridium, for expression. Additional elements of the vector can include, for example, selectable markers, e.g., kanamycin resistance or ampicillin resistance, which permit detection and/or selection of those cells transformed with the desired polynucleotide sequences.

In some embodiments the expression vector can include genes for the tolerance of a host cell to economically relevant ethanol concentrations. For example, genes such as omrA, lmrA, and lmrCD may be included in the expression vector. OmrA from wine lactic acid bacteria Oenococcus oeni and its homolog LmrA from Lactococcus lactis have been shown to increase the relative resistance of tolC(−) E. Coli by 100 to 10,000 times (Bourdineaud et al., Int'l J. Food Microbio., 92, no 1, pp. 1-14, 2004). Therefore, it may be beneficial to incorporate omrA, lmrA, and other homologous to increase the ethanol tolerance of a host cell. For example, an expression vector comprising a C. phy rnf operon can further comprise the omrA gene, the lmrA gene, the lmrCD gene, or any combination thereof. Any promoters suitable for driving the expression of a heterologous gene in a host cell can be used to drive the genes for the tolerance of a host cell, including those typically used in standard expression cassettes.

The vector used for introducing specific genes into a host microorganism may be any vector so long as it can replicate in the host microorganism. Vectors for use in the new methods can be operable as cloning vectors or expression vectors in the selected host cell. The particular vector used to transport the genetic information into the cell is also not particularly critical. Any suitable vector used for expression of recombinant proteins can be used. In certain embodiments, a vector that is capable of being inserted into the genome of the host cell is used. Numerous vectors are known to practitioners skilled in the art, and selection of an appropriate vector and host cell is a matter of choice. The vectors may, for example, be bacteriophage, plasmids, viruses, or hybrids thereof, such as those described in Maniatis et al., 1989; Ausubel et al., 1995; Miller, J. H., 1992; Sambrook and Russell, 2001. Further, the vectors described herein may be non-fusion vectors or fusion vectors.

Within each specific vector, various sites may be selected for insertion of a polynucleotide sequence of interest. These sites are usually designated by the restriction enzyme or endonuclease that cuts them. For example, the vector can be digested with a restriction enzyme matching the terminal sequence of the gene, and the vector and polynucleotide sequences can be ligated. The ligation is usually attained by using a ligase such as, for example, T4 nucleic acid ligase.

The particular site chosen for insertion of the selected nucleotide fragment into the vector to form a recombinant vector can be determined by a variety of factors. These include size and structure of the polypeptide to be expressed, susceptibility of the desired polypeptide to enzymatic degradation by the host cell components and contamination by its proteins, expression characteristics such as the location of start and stop codons, and other factors recognized by those of skill in the art. None of these factors alone absolutely controls the choice of insertion site for a particular polypeptide. Rather, the site chosen reflects a balance of these factors, and not all sites may be equally effective for a given protein.

In some embodiments, selection of a recombinant microorganism can be facilitated by resistance to antibiotics. Thus, in some embodiments, the vectors can include at least one antibiotic resistance gene. The antibiotic resistance gene can be any gene encoding resistance to any antibiotic, including without limitation, spectinomycin, kanamycin, chloramphenicol phleomycin and any analogues.

In some embodiments, the vectors described herein can include genomic nucleic acid segments for facilitating targeted integration into the host organism genome. A genomic nucleic acid segment for targeted integration can be from about ten nucleotides to about 20,000 nucleotides long. In some embodiments, a genomic nucleic acid segment for targeted integration can be about can be from about 1,000 to about 10,000 nucleotides long. In other embodiments, a genomic nucleic acid segment for targeted integration is between about 1 kb to about 2 kb long. In some embodiments, a “contiguous” piece of nuclear genomic nucleic acid can be split into two flanking pieces when the genes of interest are cloned into the non-coding region of the contiguous DNA. In other embodiments, the flanking pieces can include segments of nuclear nucleic acid sequence that are not contiguous with one another. In some embodiments, a first flanking genomic nucleic acid segment is located between about 0 to about 10,000 base pairs away from a second flanking genomic nucleic acid segment in the nuclear genome.

In some embodiments, genomic nucleic acid segments can be introduced into a vector to generate a backbone expression vector for targeted integration of any expression cassette disclosed herein into the nuclear genome of the host organism. Any of a variety of methods known in the art for introducing nucleic acid sequences can be used. For example, nucleic acid segments can be amplified from isolated nuclear genomic nucleic acid using appropriate primers and PCR. The amplified products can then be introduced into any of a variety of suitable cloning vectors, for example, by ligation. Some useful vectors include, for example, without limitation, pGEM13z, pGEMT, and pGEMTEasy (Promega, Madison, Wis.); pSTBlue1 (EMD Chemicals Inc. San Diego, Calif.); and pcDNA3.1, pCR4-TOPO, pCR-TOPO-II, pCRBlunt-II-TOPO (Invitrogen, Carlsbad, Calif.). In some embodiments, at least one nucleic acid segment from a nucleus is introduced into a vector. In other embodiments, two or more nucleic acid segments from a nucleus are introduced into a vector. In some embodiments, the two nucleic acid segments can be adjacent to one another in the vector. In some embodiments, the two nucleic acid segments introduced into a vector can be separated by, for example, between about one and thirty base pairs. In some embodiments, the sequences separating the two nucleic acid segments can contain at least one restriction endonuclease recognition site.

In various embodiments, regulatory sequences can be included in the vectors of the present invention. In some embodiments, the regulatory sequences comprise nucleic acid sequences for regulating expression of genes (e.g., a gene of interest) introduced into the nuclear genome. In various embodiments, the regulatory sequences can be introduced into a backbone expression vector. For example, various regulatory sequences can be identified from the host microorganism genome. The regulatory sequences can comprise, for example, a promoter, an enhancer, an intron, an exon, a 5′ UTR, a 3′ UTR, or any portions thereof of any of the foregoing, of a nuclear gene. Using standard molecular biology techniques, the regulatory sequences can be introduced into the desired vector. In some embodiments, the vectors comprise a cloning vector or a vector including nucleic acid segments for targeted integration. Recognition sequences for restriction enzymes can be engineered to be present adjacent to the ends of the regulatory sequences. The recognition sequences for restriction enzymes can be used to facilitate introduction of the regulatory sequence into the vector.

In some embodiments, nucleic acid sequences for regulating expression of genes introduced into the nuclear genome can be introduced into a vector by PCR amplification of a 5′ UTR, 3′ UTR, a promoter, and/or an enhancer, or a portion thereof, of one or more nuclear genes. Using suitable PCR cycling conditions, primers flanking the sequences to be amplified are used to amplify the regulatory sequences. In some embodiments, the primers can include recognition sequences for any of a variety of restriction enzymes, thereby introducing those recognition sequences into the PCR amplification products. The PCR product can be digested with the appropriate restriction enzymes and introduced into the corresponding sites of a vector.

Microorganism Hosts

A variety of different kinds of microorganisms can be used as hosts for transformation with the vectors disclosed herein. The range of microorganisms includes, for example without limitation, eukaryotic cells, such as animal cells, insect cells, fungal cells, and yeasts, and bacteria. In some embodiments, a host organism does not naturally produce ethanol. In some embodiments, the host is C. phy.

In some embodiments, the recombinant microorganism can be a cellulolytic or saccharolytic microorganism. In some embodiments, the microorganism can be Clostridium cellulovorans, Clostridium cellulolyticum, Clostridium thermocellum, Clostridium josui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Acetivibrio cellulolyticus, Bacteroides cellulosolvens, Caldicellulosiruptor saccharolyticum, Ruminococcus albus, Ruminococcusflavefaciens, Fibrobacter succinogenes, Eubacterium cellulosolvens, Butyrivibrio fibrisolvens, Anaerocellum thermophilum, Halocella cellulolytica, Thermoanaerobacterium thermosaccharolyticum, or Thermoanaerobacterium saccharolyticum.

In some embodiments, a host microorganism can be selected, for example, from the broader categories of gram-negative bacteria, such as the Xanthomonas species, and gram-positive bacteria, including members of the genera Bacillus, such as B. pumilus, B. subtilis and B. coagulans; Clostridium, for example, Cl. acetobutylicum, Cl. aerotolerans, Cl. thermocellum, Cl. thermohydrosulfuricum and Cl. thermosaccharolyticum; Cellulomonas species like C. uda; and butyrivibrio fibrisolvens. In addition to E. coli, for example, other enteric bacteria of the genera Erwinia, like E. chrysanthemi, and Klebsiella, like K. planticola and K. oxytoca, can be used. In some embodiments, the host microorganism can be Zymomonas mobilis. Similarly acceptable host organisms are various yeasts, exemplified by species of Cryptococcus like Cr. albidus, species of Monilia, Pichia stipitis and Pullularia pullulans, and Saccharomyces cerevisiae; and other oligosaccharide-metabolizing bacteria, including but not limited to Bacteroides succinogenes, Thermoanaerobacter species like T. ethanolicus, Thermoanaerobium species such as T. brockii, Thermobacteroides species like T. acetoethylicus, and species of the genera Ruminococcus (for example, R. flavefaciens), Thermonospora (such as T. fusca) and Acetivibrio (for example, A. cellulolyticus). In some embodiments, a host organism can be selected, for example, from an algae such as, for example, Amphora, Anabaena, Anikstrodesmis, Botryococcus, Chaetoceros, Chlorella, Chlorococcum, Cyclotella, Cylindrotheca, Dunaliella, Euglena, Hematococcus, Isochrysis, Monoraphidium, Nannochloris, Nannnochloropsis, Navicula, Nephrochloris, Nephroselmis, Nitzschia, Nodularia, Nostoc, Oochromonas, Oocystis, Oscillartoria, Pavlova, Phaeodactylum, Playtmonas, Pleurochrysis, Porhyra, Pseudoanabaena, Pyramimonas, Stichococcus, Synechococcus, Tetraselmis, Thalassiosira, Trichodesmium. The literature relating to microorganisms which meet the subject criteria is reflected, for example, in Biely, Trends in Biotech. 3: 286-90 (1985), in Robsen et al., Enzyme Microb. Technol. 11: 626-44 (1989), and in Beguin Ann. Rev. Microbiol. 44:219-48 (1990), each of which is herein incorporated by reference in its entirety. Appropriate transformation methodology is available for each of these different types of hosts and is described in detail below.

In some embodiments, a host microorganism can be selected by, for example, its ability to produce the proteins necessary to transport an oligosaccharide into the cell and its intracellular levels of enzymes which metabolize those oligosaccharides. Examples of such microorganisms include enteric bacteria like E. chrysanthemi and other Erwinia, and Klebsiella species such as K. oxytoca, which naturally produces a β-xylosidase, and K. planticola. Certain E. coli are attractive hosts because they transport and metabolize cellobiose, maltose and/or maltotriose. See, for example, Hall et al., J. Bacteriol. 169:2713-17 (1987).

In some embodiments, a host microorganism can be selected by, for example, screening to determine whether the tested microorganism transports and metabolizes oligosaccharides. Such screening can be accomplished in various ways. For example, microorganisms can be screened to determine which grow on suitable oligosaccharide substrates, the screen being designed to select for those microorganisms that do not transport only monomers into the cell. See, for example, Hall et al. (1987), supra. Alternatively, microorganisms could be assayed for appropriate intracellular enzyme activity, e.g., β-xylosidase activity. Growth of potential host microorganisms can be further screened for ethanol tolerance, salt tolerance, and temperature tolerance. See Alterhum et al., Appl. Environ. Microbiol. 55:1943-48 (1989); Beall et al., Biotechnol. & Bioeng. 38:296-303 (1991).

In some embodiments, a host microorganism can exhibit one or more of the following characteristics: the ability to grow in ethanol concentrations above 1.0%, 2.5%, 5.0%, 7.5%, or 10% or more ethanol, the ability to tolerate salt levels of, for example, 0.3, 0.5, 0.7 or more molar, the ability to tolerate acetate levels of, for example, 0.2, 0.3, 0.5 or more molar, and the ability to tolerate temperatures of, for example, 40° C. or more, and the ability to produce high levels of enzymes useful for cellulose, hemicellulose and pectin depolymerization with minimal protease activity. In some embodiments a host microorganism may also contain native xylanases or cellulases. In some embodiments, after introduction of expression vectors for fuel production, a certain host can produce ethanol from various saccharides tested with greater than, for examples, 90% of theoretical yield while retaining one or more useful traits above.

Transformation of Host Cells

In various embodiments, the expression vectors can be introduced, or transformed, into host microorganism cell, thereby producing a recombinant microorganism that is capable of producing a fuel when grown under a variety of fermentation conditions. Genetic engineering techniques known to those skilled in the art of transformation can be applied to carry out the methods using baseline principles and protocols unless otherwise specified.

For example, a host cell can be transformed with an expression vector comprising the C. phy rnf operon. In other embodiments, the host cell can be transformed with, for example, an expression vector comprising the C. phy rnf operon and one or more expression vectors comprising a polynucleotide sequence encoding any one or more of Pfo, acetaldehyde dehydrogenase, ethanol dehydrogenase, and hydrogenase.

A variety of different methods are known for the introduction of nucleic acids into a host cell. In various embodiments, the expression vectors can be introduced into host cells by, for example without limitation, chemical transformation, electroporation, injection, particle inflow gun bombardment, or magnetophoresis. The latter is a nucleic acid introduction technology using the processes of magnetophoresis and nanotechnology fabrication of micro-sized linear magnets (Kuehnle et al., U.S. Pat. Nos. 6,706,394 and 5,516,670).

In various embodiments, the transformation methods can be coupled with one or more methods for visualization or quantification of nucleic acid introduction to one or more microorganisms. Further, it is taught that this can be coupled with identification of any line showing a statistical difference in, for example, growth, fluorescence, carbon metabolism, isoprenoid flux, or fatty acid content from the unaltered phenotype. The transformation methods can also be coupled with visualization or quantification of a product resulting from expression of the introduced nucleic acid.

Growth, Expression, and Fuel Production

For the production of fuel, recombinant microorganisms transformed with one or more expression vectors for the production of a fuel are preferably incubated under conditions suitable for expression of the polynucleotides of interest and production of the fuel. The incubation conditions will vary depending on the host microorganism used. In certain embodiments, the incubation conditions allow fermentation. Fermentation parameters are dependent on the type of host organism used for expression of the polynucleotide(s) of interest and production of fuel.

In some instances, the concentration of the microorganism suspended in the culture medium is from about 10⁶ to about 10⁹ cells/mL, e.g., from about 10⁷ to about 10⁸ cells/mL. In some implementations, the concentration at the start of fermentation is about 10⁷ cells/mL. Clostridium phytofermentans cells can ferment both low, e.g., 0.01 mM to about 5 mM, and high concentrations of carbohydrates, and are generally not inhibited in their action at relatively high concentrations of carbohydrates, which would have adverse effects on other organisms. The same can be true for the recombinant microorganism described herein. For example, the concentration of the carbohydrate in the medium can be greater than 20 mM, e.g., greater than 25 mM, 30 mM, 40 mM, 50 mM, 60 mM, 75 mM, 100 mM, 150 mM, 200 mM, 250 mM, 300 mM, or even greater than 500 mM or more. In any of these embodiments, the concentration of the carbohydrate is generally less than 2,000 mM.

The fermentable material can be, or can include, one or more low molecular weight carbohydrates. The low molecular weight carbohydrate can be, e.g., a monosaccharide, a disaccharide, an oligosaccharide, or mixtures of these. The monosaccharide can be, e.g., a triose, a tetrose, a pentose, a hexose, a heptose, a nonose, or mixtures of these. For example, the monosaccharide can be arabinose, glyceraldehyde, dihydroxyacetone, erythrose, ribose, ribulose, xylose, glucose, galactose, mannose, fucose, fructose, sedoheptulose, neuraminic acid, or mixtures of these. The disaccharide can be, e.g., sucrose, lactose, maltose, gentiobiose, or mixtures of these.

In some embodiments, the low molecular weight carbohydrate is generated by breaking down a high molecular weight polysaccharides (e.g., cellulose, xylan or other components of hemicellulose, pectin, and/or starch). This technique can be advantageously and directly applied to waste streams, e.g., waste paper (e.g., waste newsprint and waste cartons). In some instances, the breaking down is done as a separate process, and then the low molecular weight carbohydrate utilized in culturing the new recombinant microorganism described herein. In other instances, the high molecular weight carbohydrate is added directly to the medium, and is broken down into the low molecular weight carbohydrate in-situ. In some implementations, this is done chemically, e.g., by oxidation, base hydrolysis, and/or acid hydrolysis. Chemical hydrolysis has been described by Bjerre, Biotechnol. Bioeng., 49:568, 1996, and Kim et al., Biotechnol. Prog., 18:489, 2002.

Various media for growing a variety of microorganisms are known in the art. Growth media may be minimal/defined or complete/complex. Fermentable carbon sources can include any biomass material, including pretreated (e.g., by cutting, chopping, or wetting), or non-pretreated feedstock containing cellulosic, hemicellulosic, and/or lignocellulosic material. The various types of biomass include plant biomass and municipal waste biomass (residential and light commercial refuse with recyclables such as metal and glass removed).

The terms “plant biomass” and “lignocellulosic biomass” refer to any plant-derived organic matter (woody or non-woody) available for energy on a sustainable basis. Plant biomass can include, but is not limited to, agricultural crop wastes and residues such as corn stover, wheat straw, rice straw, sugar cane bagasse, and the like. Plant biomass further includes, but is not limited to, trees, woody energy crops, wood wastes and residues such as softwood forest waste, sawdust, paper and pulp industry waste streams, wood fiber, and the like. Additionally grass crops, such as switchgrass and the like have potential to be produced on a large-scale as another plant biomass source. Other types of plant biomass include yard waste (e.g., grass clippings, leaves, tree clippings, and brush) and vegetable processing waste.

“Lignocellulosic materials” include cellulose and a percentage of lignin, e.g., at least about 0.5 percent by weight to about 60 percent by weight or more lignin. These materials include plant biomass such as, but not limited to, non-woody plant biomass, cultivated crops, such as, but not limited to, grasses, for example, but not limited to, C3 or C4 grasses, such as switchgrass, cord grass, rye grass, miscanthus, or a combination thereof, or sugar processing residues such as bagasse, or beet pulp, agricultural residues, for example, soybean stover, corn stover, rice straw, rice hulls, barley straw, corn cobs, wheat straw, canola straw, rice straw, oat straw, oat hulls, corn fiber, wood pulp fiber, sawdust, hardwood, softwood, or a combination thereof. Further, the lignocellulosic materials may include cellulosic waste material such as, but not limited to, newsprint, recycled paper, and cardboard.

In particular implementations, the lignocellulosic material is obtained from trees, such as Coniferous trees, e.g., Eastern Hemlock (Tsuga canadensis), Maidenhair Tree (Ginkgo bilboa), Pencil Cedar (Juniperus virgineana), Mountain Pine (Pinus mugo), Deodar (Cedrus deodara), Western Red Cedar (Thula plicata), Common Yew (Taxus baccata), Colorado Spruce (Picea pungens); or Deciduous trees, e.g., Mountain Ash (Sorbus), Gum (Eucalyptus gunnii), Birch (Betula platyphylla), or Norway Maple (Acer platanoides), can be utilized. Poplar, Beech, Sugar Maple and Oak trees may also be utilized.

In some instances, the recombinant microorganisms can ferment lignocellulosic materials directly without the need to remove lignin. However, in certain embodiments, it is useful to remove at least some of the lignin from lignocellulosic materials before fermenting. For example, removal of the lignin from the lignocellulosic materials can make the remaining cellulosic material more porous and higher in surface area, which can, e.g., increase the rate of fermentation and ethanol yield. The lignin can be removed from lignocellulosic materials, e.g., by sulfite processes, alkaline processes, or by Kraft processes. Such process and others are described in Meister, U.S. Pat. No. 5,138,007, and Knauf et al., International Sugar Journal, 106:1263, 147-150 (2004).

These biomass, e.g., cellulosic, materials can be pretreated before being added to a culture medium. In some cases, methods of processing begin with a physical preparation of the biomass material, e.g., size reduction of raw biomass materials, such as by cutting, grinding, shearing, or chopping. In some cases, loose materials (e.g., recycled paper or switchgrass) are prepared by shearing or shredding. Screens and/or magnets can be used to remove oversized or undesirable objects such as, for example, rocks or nails from the feed stream.

In some embodiments, the biomass material to be processed is in the form of a fibrous material that includes fibers provided by shearing a fiber source. For example, the shearing can be performed with a knife system, such as a rotary knife cutter system. If desired, the biomass can be cut, e.g., with a shredder, prior to the shearing. As an alternative to shredding, the biomass material can be reduced in size by cutting to a desired size using a guillotine cutter. In some embodiments, the shearing of the biomaterial and the passing of the resulting first fibrous material through a screen are performed concurrently. The shearing and the screening can also be performed in a batch-type process.

Once the biomass material is sufficiently pretreated and added to a culture medium, additional nutrients can be, but need not always be, added to the culture medium. Such additional nutrients include nitrogen-containing compounds such as proteins, hydrolyzed proteins, ammonia, urea, nitrate, nitrite, soy, soy derivatives, casein, casein derivatives, milk powder, milk derivatives, whey, hydrolyze yeast, autolyzed yeast, corn steep liquor, corn steep solids, monosodium glutamate, and/or other fermentation nitrogen sources, vitamins, and/or mineral supplements.

In some embodiments additional culture medium components include buffers, e.g., NaHCO₃, NH₄Cl, NaH₂PO₄.H₂O, K₂HPO₄, and KH₂PO₄; electrolytes, e.g., KCl, and NaCl; growth factors; surfactants; and chelating agents. Additional growth factors can include, e.g., biotin, folic acid, pyridoxine-HCl, riboflavin, urea, yeast extracts, thymine, tryptone, adenine, cytosine, guanosine, uracil, nicotinic acid, pantothenic acid, B12 (Cyanocobalamine), p-aminobenzoic acid, and thioctic acid. Minerals can include, e.g., MgSO₄, MnSO₄.H₂O, FeSO₄.7H₂O, CaCl₂.2H₂O, CoCl₂.6H₂O, ZnCl₂, CuSO₄.5H₂O, AlK(SO₄)₂.12H₂O, H₃BO₃, Na₂MoO₄, NiCl₂.6H₂O, and NaWO₄.2H₂O. Chelating agents can include, e.g., nitrilotriacetic acid. Surfactants can include, e.g., polyethylene glycol (PEG), polypropylene glycol (PPG), copolymers of PEG and PPG, and polyvinylalcohol.

The temperature of the medium is generally maintained at less than about 45° C., e.g., less than about 42° C. (e.g., between about 34° C. and 38° C., or about 37° C.). In general, the medium is maintained at a temperature above about 5° C., e.g., above about 15° C. The pH of the medium is generally maintained below about 9.5, e.g., between about 6.0 and 9.0, or between about 8 and 8.5. Generally, during fermentation, the pH of the medium typically does not change by more than 1.5 pH units. For example, if the fermentation starts at a pH of about 7.5, it typically does not go lower than pH 6.0 at the end of the fermentation, which is within the growth range of the cells. The pH of the fermentation broth can be adjusted using neutralizing agents such as calcium carbonate or hydroxides. The selection and incorporation of any of the above fermentative methods is highly dependent on the host strain and the preferred downstream process.

In some embodiments, one or more additional lower molecular weight carbon sources can be added or be present such as glucose, sucrose, maltose, corn syrup, and lactic acid. In some embodiments, one possible form of growth media can be modified Luria-Bertani (LB) broth (with 10 g Difco tryptone, 5 g Difco yeast extract, and 5 g sodium chloride per liter). In other embodiments of the invention, cultures of constructed strains of the invention can be grown in NBS mineral salts medium and supplemented with 2% to 20% sugar (w/v) or either 5% or 10% sugar (glucose or sucrose). The microorganisms can be grown in or on NBS mineral salts medium.

Fuel production can be observed by standard methods known to those skilled in the art. In some embodiments, fermentors that include a medium that includes the recombinant microorganisms dispersed therein are configured to continuously remove a fermentation product, such as ethanol. In some embodiments, the concentration of the desired product remains substantially constant, or within about twenty five percent of an average concentration, e.g., measured after 2, 3, 4, 5, 6, or 10 hours of fermentation at an initial concentration of from about 10 mM to about 25 mM. In some embodiments, any biomass material or mixture described herein is continuously fed to the fermentors.

Clostridium phytofermentans cells adapt to relatively high concentrations of ethanol, e.g., 7 percent by weight or higher, e.g., 12.5 percent by weight. Thus, the same can be true for the transformed microorganisms described herein. These microorganisms can be grown in an ethanol rich environment prior to fermentation, e.g., 7 percent ethanol, to adapt the cells to even higher concentrations of ethanol, e.g., 20 percent. In some embodiments, the microorganisms are adapted to successively higher concentrations of ethanol, e.g., starting with 2 percent ethanol, then 5 percent ethanol, and then 10 percent ethanol.

In some embodiments, growth and production of the recombinant microorganisms disclosed herein can be performed in normal batch fermentations, fed-batch fermentations, or continuous fermentations. In certain embodiments, it is desirable to perform fermentations under reduced oxygen or anaerobic conditions for certain hosts. In other embodiments, fuel production can be performed with oxygen; and, optionally with the use of air-lift or equivalent fermentors. In some embodiments, the recombinant microorganisms are grown using batch cultures. In some embodiments, the recombinant microorganisms are grown using bioreactor fermentation. In some embodiments, the growth medium in which the recombinant microorganisms are grown is changed, thereby allowing increased levels of fuel production. The number of medium changes may vary.

There are two basic approaches to produce fuels such as ethanol or hydrogen from biomass on a large scale using the recombinant microorganisms described herein. In the first method, one first hydrolyzes, e.g., using chemical or enzymatic pretreatment, a biomass material that includes high molecular weight carbohydrates to lower molecular weight carbohydrates, and then ferments the lower molecular weight carbohydrates using the recombinant microorganisms to produce the fuel. In the second method, one ferments the biomass material itself without chemical and/or enzymatic pretreatment. For more details on large-scale production of fuels, see, e.g., U.S. Patent Application No. 2007/0178569.

### EXAMPLES

The following examples are by way of illustration and not by way of limitation.

### Example 1

### Abundance of mRNA Expression Levels

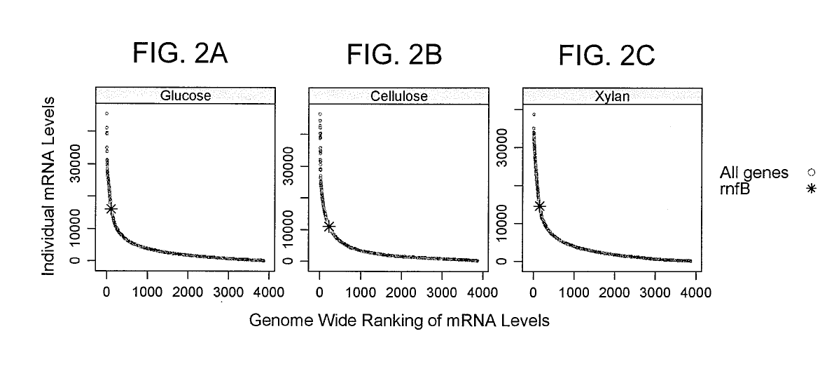

This example describes testing of mRNA expression levels of the rnfB gene. C. phy was grown on fifteen different carbon sources, and the expression levels of the C. phy rnfB gene were determined from microarray experiments and plotted as a function of genome-wide mRNA ranking: glucose (FIG. 2A), cellulose (FIG. 2B), and xylan (FIG. 2C). The rnf genes were expressed at very high levels (in the top 2-5% of all genes in the genome) during growth on all fifteen substrates tested (Glucose, Galactose, Fucose, Rhamnose, D-Arabinose, L-Arabinose, Xylose, Mannose, Galacturonic acid, Cellobiose, Cellulose, Xylan, Pectin, Laminarin, and Yeast extract). The expression of the rnfB gene and those listed in a Table 1 herein are all highly correlated and highly expressed. These results support a central role of the rnf genes in C. phy metabolism as outlined in the diagram in FIG. 1.

C. phytofermentans ISDg was cultured in anaerobic medium GS-2CB. Growth on a single carbon-source utilized an anaerobic medium derived from GS-2CB and containing the following (g/l): yeast extract, 6.0; urea, 2.1; KH2PO4, 4.0; Na2HPO4, 6.5; trisodium citrate dihydrate, 3.0; L-cysteine hydrochloride monohydrate, 2.0; resazurin, 1; with pH adjusted to 7.0 using KOH. This medium was supplemented with 0.3% (wt/vol) of the specific substrate added as a filter-sterilized solution to the sterile medium. Broth cultures were incubated at 30° C. under anaerobic conditions (100% N₂)(Hungate, Methods Microbiol., 3:117-131, 1969). Growth was determined spectrophotometrically by monitoring changes in optical density at 660 nm.

RNA was purified from mid-exponential phase cultures. Samples were flash-frozen by immersion in liquid nitrogen. The cells were collected by centrifugation for 5 minutes at 8,000 rpm at 4° C. Harvested cells were resuspended in 100 μl in TE buffer pH 8 (EMD Chemicals) containing 2 mg/ml lysozyme (Sigma-Aldrich) and incubated at 37° C. for 40 minutes. The total RNA was isolated using RNeasy® RNA purification kit (QIAGEN) according to manufacturer's instructions. Contaminating DNA in total RNA preparations was removed with RNAse-free DNase I (QIAGEN). The RNA concentration was determined by absorbance at 260/280 nm using a Nanodrop.

Our C. phytofermentans custom Affymetrix microarray design enables the measurement of the expression level of all open reading frame (ORFs), estimation of the 5′ and 3′ untranslated regions of mRNA, operon determination, sRNA discovery, and discrimination between alternative gene models (primarily differing in the selection of the start codon). Putative protein coding sequences were identified using GeneMark® (Besemer et al., GeneMark: web software for gene finding in prokaryotes, eukaryotes and viruses. Nucleic Acids Res 33: W451-454.34, 2005) and Glimmer (Delcher et al., Identifying bacterial genes and endosymbiont DNA with Glimmer,” Bioinformatics, 23:673-679, 2007).

The union of these two predictions was used as our expression set. If two proteins differed in their N-terminal region, the smaller of the two proteins was used for transcript analysis, but the extended region was represented by probes in order to define the actual N-terminus. This array design resulted in the inclusion of all proteins represented in the GenBank record as well as additional ORFs not found in the GenBank record, because we were interested in ORFs even if they had a low probability of representing functional proteins. The remaining probes were used to map expression in intergenic regions. These probes represent both DNA strands and were tiled with a 1-nucleotide gap. Standard Affymetrix array design protocols were followed to ensure each probe was unique in order to minimize cross hybridization. The array design was implemented on a 49-5241 format Affymetrix GeneChip® with 11 μg features.

Ten μg g total RNA from each sample was used as template to synthesize labeled cDNAs using Affymetrix GeneChip® DNA Labeling Reagent Kits. The labeled cDNA samples were hybridized with our Affymetrix GeneChip® Arrays according to Affymetrix guidelines. The hybridized arrays were scanned with a GeneChip® Scanner 3000. The resulting raw spot image data files were processed into pivot, quality report, and normalized probe intensity files using Microarray Suite version 5.0 (MAS 5.0). In addition, expression values were calculated using the Custom Array Analysis Software (CAAS) package (on the Internet at sourceforge.net/projects/caas-microarray) that implements the Robust Multichip Average method (Irizarry et al., “Summaries of Affymetrix GeneChip probe level data,” Nucleic Acids. Res., 31:e15, 2003). The individual microarray files (GSM333247-52) and the normalized gene summary values for the complete data set (GSE13194) have been deposited in Gene Expression Omnibus (GEO) database at the National Center for Biotechnology (ncbi.nlm.nih.gov/geo/).

The quality of the microarray data sets were analyzed using probe-level modeling procedures provided by the affyPLM package (Bolstad et al., “Quality Assessment of Affymetrix GeneChip Data,” in: Gentleman et al., editors, Bioinformatics and Computational Biology Solutions Using R and Bioconductor (Heidelberg: Springer. pp. 33-47, 2005)) in BioConductor (Gentleman et al., “Bioconductor: open software development for computational biology and bioinformatics,” Genome Biol., 5:R80, 2004). No image artifacts due to array manufacturing or processing were observed. Microarray backgrounds were within the typical 20-100 average background values for Affymetrix GeneChip®. In summary, all quality control checks indicated that the RNA purification, cDNA synthesis and labeling and hybridization procedures adapted for use in C. phytofermentans resulted in high quality data.

The expression levels of the C. phy rnfB gene (shown as a * in the graphs) were plotted as a function of genome-wide mRNA ranking for three carbon sources: glucose (FIG. 2A), cellulose (FIG. 2B), and xylan (FIG. 2C). The rnf genes were expressed at very high levels (in the top 2-5% of all genes in the genome) during growth on these three carbon sources, as well as the other twelve substrates (data not shown). The expression of the rnfB gene and those listed in a Table 1 herein are all highly correlated and highly expressed. These results support a central role of the rnf genes in C. phy metabolism as outlined in the diagram in FIG. 1.

### Example 2

### Preparation of an Expression Vector for the Production of a Fuel

This Example illustrates the preparation of one possible expression vector for the production of a fuel, a C. phy rnf operon expression vector.

Polymerase chain reaction (PCR) is used for amplification of the C. phy rnf operon sequence from C. phy genomic DNA and for the simultaneous introduction of restriction enzyme sites at the 5′ and 3′ ends, respectively. These sites allow for subcloning the C. phy rnf operon into a vector.

PCR is performed using primers containing sequences from the 5′ and 3′ end of the C. phy rnf operon sequence and desired restriction endonuclease sites. PCR conditions are as follows: Total reaction vol. of about 50 μl, about 1 μg of C. phy genomic DNA as template, about 4 Units of Vent_{R}® polymerase, a final concentration of about 0.5 μM for each primer, and about 300 μM of each dNTP. Reaction conditions are provided as follows: on an Eppendorf® Mastercycler®: Initial denaturation at 94° C. for 2 minutes, followed by 35 cycles of 10 seconds denaturation at 94° C., 1 minute annealing at 47° C., and 4 minutes extension at 68° C.; finally, hold at 4° C.

The amplified C. phy rnf operon polynucleotide is digested with the appropriate restriction enzymes and then ligated into digested vector. The vector can have a selection cassette which is removed by the insertion of the C phy rnf operon sequence, thereby facilitating selection of vectors containing the C phy rnf operon polynucleotide.

Plasmid/PCR product cleanup kits and Taq DNA polymerase are commercially available from, for example, Qiagen®. Restriction enzymes, Vent_{R}® Polymerase and T4 DNA ligase are commercially available from, for example, New England Biolabs®.

### Example 3

### Transformation and Screening for Stable Ethanol Production

This Example illustrates the construction of a stable microorganism line for production of ethanol.

Following creation of the C phy rnf operon expression vector, a host microorganism is transformed and screened sequentially for positive transformants. Transformants are screened on appropriate medium. Screening is performed, for example, via serial streaking of single colonies coupled with both an initial PCR-based assay used for probing the C. phy rnf operon cassette. A seed reactor based assay is performed for determination of stability of ethanol generation given the absence of selective pressure.

The PCR assay consists of at least two PCR reactions per sample, probing for the presence of the (1) the selection cassette, and (2) C. phy rnf operon cassette. Each of the reactions comprising the PCR assay share a common upstream primer that recognizes a site outside of the site of C. phy rnf operon cassette insertion, while each reaction is defined by the downstream primer that is specific for each possible genetic construct. All PCR reactions are formulated as described in the Qiagen® Taq Polymerase Handbook in the section for long PCR products, modified by the exclusion of any high fidelity polymerase. The cycling program is as follows: Initial denaturation at 94° C. for 3 minutes, followed by 35 cycles of 10 seconds denaturation at 94° C., 1 minute annealing at 48° C., and 3.5 minutes extension at 68° C.; a final 3 minutes extension at 68° C., hold at 4° C.

To perform the PCR assay on a given microorganism sample, genomic DNA is prepared for use as a template in the above PCR reaction. For testing a liquid culture, an amount of culture, for example, 5 μl, is spotted onto an appropriate substrate. For testing cultures streaked on solid media, multiple colonies are lifted from the plate, streaked on the inside of a tube, and resuspended in media via mixing; an amount of the suspension, for example, 5 μl, is then spotted onto an appropriate substrate, as above. The genomic DNA for use as a template is then prepared for the PCR assay.

The primary seed reactor based assay is used to screen colonies that are shown to be completely segregated for the C. phy rnf operon cassette for stable ethanol production. Seed reactors are inoculated with multiple colonies from a plate of a given recombinant microorganism. The recombinant microorganism cells are grown, collected by centrifugation, and resuspended in a fresh seed reactor at an initial density. This constitutes the first experimental reactor in a series of five runs. The reactor is run for a set period of time, at which point the cells are again collected by centrifugation and used to inoculate the second experimental reactor in the series to the above density. Of course, only a subset of the total cell biomass is used for this serial inoculation while the rest is discarded or prepared as a glycerol stock.

Each day of a particular run, the density is recorded, and an aliquot is taken for an ethanol concentration assay (the “before” aliquot). The cells are then washed by collection via centrifugation (as above), the supernatant is discarded, the cells are resuspended by vortexing the entire pellet in fresh media, and are then returned to the seed reactor. The density is again recorded and another aliquot is taken for an ethanol concentration assay (the so called “after” aliquot). After isolation of a stable ethanol producing isolate, the PCR-based assay can be performed a final time for confirmation.

### Example 4

### Batch Growth Experiments

This Example illustrates batch growth experiments for productivity and stability studies.

A parallel batch culture system (for example, six 100 mL bioreactors) is established to grow the ethanol-producing host microorganism strains developed. The seed cultures are started from a plate, and exponentially growing cells from a seed culture are inoculated into the reactors. Standard liquid media is used for the all the experiments. Compressed air is sparged to provide CO₂ and remove the oxygen produced by recombinant microorganisms. Semi-batch operation mode is used to test the ethanol production. The total cell growth period is, for example, about 20 days. Batch cultures are conducted for about 4 days, and then terminated. The cells are spun down by centrifugation, resuspended in a reduced volume, and an aliquot is used to inoculate a bioreactor with fresh media.

### Example 5

### Ethanol Concentration Assay

For determination of ethanol concentration of a liquid culture, an aliquot of the culture is taken, spun down, and an appropriate volume of the supernatant is placed in a fresh tube and stored at −20° C. until the assay is performed. Given the linear range of the spectrophotometer and the sensitivity of the ethanol assay, dilution of the sample (up to, for example, 20 fold) may be occasionally required. In this case, an appropriate volume is added to the fresh tube, to which the required volume of clarified supernatant is added. This solution is used directly in the ethanol assay. Upon removal from −20° C. and immediately before performing the assay, the samples are spun down a second time at to assist in sample thawing.

The Boehringer Mannheim/r-Biopharm® enzymatic ethanol detection kit is used for ethanol concentration determination. Briefly, this assay exploits the action of ethanol dehydrogenase and acetaldehyde dehydrogenase in a phosphate-buffered solution of the NAD⁺ cofactor, which upon the addition of ethanol causes a conversion of NAD⁺ to NADH. Concentration of NADH is determined by light absorbance at 340 nm (A₃₄₀) and is then used to determine ethanol concentration. The assay was performed as given in the instructions, with the following modifications. Media is used as a blank control.

### Other Embodiments

The foregoing description and Examples detail certain specific embodiments of the invention and describes the best mode contemplated by the inventors. It will be appreciated, however, that no matter how detailed the foregoing may appear, the invention can be practiced in many ways and the invention should be construed in accordance with the appended claims and any equivalents thereof.

What is claimed is:

1. An isolated polynucleotide that encodes a polypeptide that modulates fuel production in C. phytofermentans.

2. The polynucleotide of claim 1, wherein the polynucleotide comprises a nicotinamide adenine dinucleotide (NADH) ferredoxin oxidoreductase (Nfo) subunit.

3. The polynucleotide of claim 1, wherein the polynucleotide comprises a C. phytofermentans rnf operon.

4. The polynucleotide of claim 1, wherein the polynucleotide comprises a nucleic acid sequence corresponding to a region of the C. phytofermentans chromosome extending from about position 259945 to about position 265175.

5. The polynucleotide of claim 1, wherein the polynucleotide comprises at least one nucleic acid sequence selected from the group consisting of SEQ ID NO: 1, SEQ ID NO:2, SEQ ID NO:3, SEQ ID NO:4, SEQ ID NO:5, and SEQ ID NO:6.

6. The polynucleotide of claim 2, wherein the Nfo subunit is selected from the group consisting of RnfC, RnfD, RnfG, RnfE, RnfA, and RnfB.

7. The polynucleotide of claim 1, wherein the polynucleotide comprises a nucleic acid sequence encoding RnfC, RnfD, RnfG, RnfE, RnfA, and RnfB.

8. The polynucleotide of claim 1, wherein the polynucleotide further comprises a nucleic acid sequence encoding an enzyme selected from the group consisting of pyruvate ferredoxin oxidoreductase (Pfo), acetaldehyde dehydrogenase, ethanol dehydrogenase, and hydrogenase.

9. An expression cassette that enables an organism to produce a fuel, the expression cassette comprising an isolated polynucleotide that encodes at least one polypeptide that modulates fuel production in C. phytofermentans.

10. The expression cassette of claim 9, wherein the polynucleotide comprises a nucleic acid sequence encoding a nicotinamide adenine dinucleotide (NADH) ferredoxin oxidoreductase (Nfo) subunit.

11. The expression cassette of claim 9, wherein the polynucleotide comprises a C. phy rnf operon.

12. The expression cassette of claim 10, wherein the Nfo subunit is selected from the group consisting of RnfC, RnfD, RnfG, RnfE, RnfA, and RnfB.

13. The expression cassette of claim 9, wherein the polynucleotide comprises a nucleic acid sequence encoding RnfC, RnfD, RnfG, RnfE, RnfA, and RnfB.

14. The expression cassette of claim 9, wherein the polynucleotide further comprises a nucleic acid sequence encoding an enzyme selected from the group consisting of pyruvate ferredoxin oxidoreductase (Pfo), acetaldehyde dehydrogenase, ethanol dehydrogenase, and hydrogenase.

15. The expression cassette of claim 9, wherein the polynucleotide further comprises a sequence encoding a selectable marker.

16. An isolated microorganism comprising a heterologous polynucleotide encoding at least one polypeptide that encodes a polypeptide that modulates fuel production in C. phytofermentans.

17. The microorganism of claim 16, wherein the microorganism ferments cellulose-containing biomass to produce at least one fuel.

18. The microorganism of claim 16, wherein the heterologous polynucleotide comprises a nucleic acid sequence corresponding to a gene from a C. phytofermentans metabolic pathway.

19. The microorganism of claim 16, wherein the heterologous polynucleotide comprises a nucleic acid sequence encoding a nicotinamide adenine dinucleotide (NADH) ferredoxin oxidoreductase (Nfo) subunit.

20. The microorganism of claim 16, wherein the heterologous polynucleotide comprises the C. phy rnf operon.

21. The microorganism of claim 16, wherein the heterologous polynucleotide comprises a nucleic acid sequence encoding RnfC, RnfD, RnfG, RnfE, RnfA, and RnfB.

22. The microorganism of claim 19, wherein the Nfo subunit is selected from the group consisting of RnfC, RnfD, RnfG, RnfE, RnfA, and RnfB.

23. The microorganism of claim 16, wherein the heterologous polynucleotide further comprises a nucleic acid sequence encoding an enzyme selected from the group consisting of pyruvate ferredoxin oxidoreductase (Pfo), acetaldehyde dehydrogenase, ethanol dehydrogenase, and hydrogenase.

24. The microorganism of claim 16, wherein the heterologous polynucleotide further comprises a sequence encoding a selectable marker.

25. The microorganism of claim 16, wherein the microorganism is a prokaryote or eukaryote.

26. The microorganism of claim 16, wherein the microorganism is selected from a group consisting of Escherichia, Zymomonas, Saccharomyces, Candida, Pichia, Streptomyces, Bacillus, Lactobacillus, and Clostridium.

27. The microorganism of claim 16, wherein the microorganisms is selected from the group consisting of Clostridium cellulovorans, Clostridium cellulolyticum, Clostridium thermocellum, Clostridium josui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Acetivibrio cellulolyticus, Bacteroides cellulosolvens, Caldicellulosiruptor saccharolyticum, Ruminococcus albus, Ruminococcus flavefaciens, Fibrobacter succinogenes, Eubacterium cellulosolvens, Butyrivibrio fibrisolvens, Anaerocellum thermophilum, Halocella cellulolytica, Thermoanaerobacterium thermosaccharolyticum, and Thermoanaerobacterium saccharolyticum.

28. The microorganism of claim 16, wherein the microorganism produces a fuel in recoverable quantities greater than about 10 mM fuel after a 5 day fermentation.

29. The microorganism of claim 16, wherein said fuel is ethanol.

30. A method for producing fuel, the method comprising culturing a microorganism of claim 16 in a culture medium.

31. The method of claim 30, wherein the microorganism comprises a polynucleotide encoding a nicotinamide adenine dinucleotide (NADH) ferredoxin oxidoreductase (Nfo) subunit.

32. The method of claim 31, wherein the Nfo subunit is selected from the group consisting of RnfC, RnfD, RnfG, RnfE, RnfA, and RnfB.

33. The method of claim 30, wherein the polynucleotide comprises an rnf operon.

34. The method of claim 30, wherein the heterologous polynucleotide comprises a nucleic acid sequence encoding RnfC, RnfD, RnfG, RnfE, RnfA, and RnfB.

35. The method of claim 30, wherein the heterologous polynucleotide further comprises a nucleic acid sequence encoding an enzyme selected from the group consisting of pyruvate ferredoxin oxidoreductase (Pfo), acetaldehyde dehydrogenase, ethanol dehydrogenase, and hydrogenase.

36. The method of claim 30, wherein the microorganism is a prokaryote or eukaryote.

37. The method of claim 30, wherein the microorganism is selected from a group consisting of Escherichia, Zymomonas, Saccharomyces, Candida, Pichia, Streptomyces, Bacillus, Lactobacillus, and Clostridium.

38. The method of claim 30, wherein the microorganisms is selected from the group consisting of Clostridium cellulovorans, Clostridium cellulolyticum, Clostridium thermocellum, Clostridium josui, Clostridium papyrosolvens, Clostridium cellobioparum, Clostridium hungatei, Clostridium cellulosi, Clostridium stercorarium, Clostridium termitidis, Clostridium thermocopriae, Clostridium celerecrescens, Clostridium polysaccharolyticum, Clostridium populeti, Clostridium lentocellum, Clostridium chartatabidum, Clostridium aldrichii, Clostridium herbivorans, Acetivibrio cellulolyticus, Bacteroides cellulosolvens, Caldicellulosiruptor saccharolyticum, Ruminococcus albus, Ruminococcus flavefaciens, Fibrobacter succinogenes, Eubacterium cellulosolvens, Butyrivibrio fibrisolvens, Anaerocellum thermophilum, Halocella cellulolytica, Thermoanaerobacterium thermosaccharolyticum, and Thermoanaerobacterium saccharolyticum.

39. The method of claim 30, wherein the microorganism produces a fuel in recoverable quantities greater than about 10 mM fuel after a 5 day fermentation.

40. The method of claim 30, wherein the fuel is hydrogen or ethanol.

41. The method of claim 30, wherein the culturing is performed in normal batch fermentation, fed-batch fermentation, or continuous fermentation.

42. The method of claims 30, where the culture medium comprises pretreated or non-pretreated feedstock.

43. The method of claim 42, wherein the feedstock comprises cellulosic, hemicellulosic, and/or lignocellulosic material.

44. The method of claim 43, wherein the culture medium comprises glucose, cellulose, xylan, or a combination thereof.

45. The method of claim 43, wherein the fuel is ethanol.


SEQUENCE LISTING (18 sequences)

SEQ ID NO: 1 - protein, 439 aa, Clostridium phytofermentans
MAAGTFKGGIHPYEGKELTKDKPTTLLLPKGDLVYPMSQHIGNPAKPIVAKGDKVLVGQKIGEADGVVSACIISSVSGTVKAVEPRLNVAGTMVESIVVENDNAYTQVEGFGVERDYETLKKEQIRSIIKEAGIVGMGGAGFPTHIKLTPKDDSAIDYLIINGSECEPYLTSDYRMMLEETNRLIKGIKITLRLFENAKAIIAVEDNKPEAISMLTHALRNENRIELKVIKTKYPQGAERVLIYAITGRKMNSTMLPSDIGCIVNNVDTMISVCRAVAENTPLIKRVVTVSGDAVKNQGNFIVLTGTNYSELVEAVGGFSAKPAKLISGGPMMGLALYSLDIPVTKTSSALLAFASDEVADMEEGPCIRCGRCVEVCPGRIVPQKLMEFAERFDDKGFEGLNGMECCECGCCSYICPAGRHLTQAFKQSKRSILNERKK

SEQ ID NO: 2 - protein, 336 aa, Clostridium phytofermentans
MKDMYNVSASPHVRSGVTTAQIMRDVAIALMPACLFGIYQFGFSAFLVLLVSVTSCVVSEFLYERLMKHPYRPYECSALVTGLLIGMNMPATIPVWIPMVGGVFAIIVVKQLYGGLGQNFMNPALAARCFLSICFTSRMTTFAVDAFTNSGTSSRTLYLFNYGYAGLDGVSGATPLAAMKASEAAPSLLDMFFGFHGGVIGETSAMMLLIGACYLLYRRIISLRIPLTYIATFAVFIILFSGKGFDVEYVLAQILGGGLILGAFFMATDYVTCPITKYGQILFGVCLGALTGLFRVFGGSAEGVSYAIIFCNLLVPLIEKITMPRGFGMGGKKLAK

SEQ ID NO: 3 - protein, 218 aa, Clostridium phytofermentans
MQNKKKSTIIKDAIALFAITLVAAVALGFVYEITKDPIAEAEAKAKAKAYSMVFADAKLVDDKNEDVNAKVDSSKEFLTSQGFTSSTINEVCIAKDEAGNALGFVMTLTSSAGYGGDIKFTMGVKADGTLTSIEIISMNETSGLGAKANDDSFKGQYSDKNVDSFKVIKSAESKTGDDQINAISGATITSSAVTGTVNAGLAFANDLLENGVGGVTHE

SEQ ID NO: 4 - protein, 253 aa, Clostridium phytofermentans
MSKALERIYNGVIKENPTFVLMLGMCPTLAVTTSAINGVGMGLTTTAVLIMSNMLISMLRKAIPDKVRMPAFIVVVASFVTIVQLLLQAYLPSLNDSLGIYIPLIVVNCIILGRAEAYASKYPVYPSIFDGVGMGLGFTVGLTLIGLFREILGAGTAFGFSIMPDSYEPFSIFVLAPGAFFVLAMLTALQNKLKLKSATNVPMADKLACGGNCSSCSGSACHSNHELLDSVKEEATKKAAAEKARAANQTEKK

SEQ ID NO: 5 - protein, 191 aa, Clostridium phytofermentans
MKELLLVLIAAALVNNVVLSRFLGLCPFLGVSKKISTAAGMGGAVIFVITIASALCSVIYDVVLVPLDLKYMNTIVFIILIAALVQFIEMFLKKFSPGLYNALGVYLPLITTNCAVLGVAIDNVQKGNGFVISVVYGAGTAIGFLIAIVIMAGVRERIENNNVTKSFQGSPIVLITAGLMSIAFMGFAGLL

SEQ ID NO: 6 - protein, 282 aa, Clostridium phytofermentans
MTNLALFDLLSNTGVLAFNMQGLITAAAIVGGVGLIIGILLGLAAKVFEVEVDERELIVRDLLPGNNCGGCGYPGCDGLAKAIAAGEAPVSGCPVASAEIHAKIGEVMGTEAIESERNVAFVKCNGTCDKTNVKYHYTGTPDCKKISTVPGNGEKTCIYGCMGYGSCVRACAFDAIHVVNGIAVVDKEKCVACGKCITACPNDLIEFVPVSSTCKVQCNSKDKGKDVNAACSVGCIGCMMCVKVCESDAVTVTNNLAHIDYSKCTHCGKCAEKCPRKIITIA

SEQ ID NO: 7 - protein, 396 aa, Clostridium phytofermentans
MARFTLPRDLYHGKGSLAELKNLTGKKAIIVVGGGSMKRFGFLDRAIDYIKEAGMEVSLFENVEPDPSVETVMKGAAAMREFEPDWIISMGGGSPIDAAKAMWAFYEYPDTTFEDLIVPFNFPTLRTKAKFCAIPSTSGTATEVTAFSVITDYHKGIKYPLADFNITPDVAIVDPDLAETMPAKLTAHTGMDAMTHAVEAYVSTLHCDYTDPLAMHAIRMVHEYLKSSYDGNMDARDKMHNAQCLAGMAFSNALLGIVHSMAHKTGAAYSGGHIVHGCANAMYLPKVIKFNSKNEDAAKRYAEIATALFLKGNTTTELVDALIEELNQMNRSLNIPSCIKEYENGIIDEKEFLEKLPEVAANAISDACTGSNPRIPTQEEMEKLLKACFYNEEITF

SEQ ID NO: 8 - protein, 872 aa, Clostridium phytofermentans
MTKKVELQTTGLVDSLEALTAKFRELKEAQELFATYTQEQVDKIFFAAAMAANQQRIPLAKMAVEETGMGIVEDKVIKNHYAAEYIYNAYKDTKTCGVVEEDPSFGIKKIAEPIGVVAAVIPTTNPTSTAIFKTLLCLKTRNAIIISPHPRAKNCTIAAAKVVLDAAVAAGAPAGIIGWIDVPSLELTNEVMKNADIILATGGPGMVKAAYSSGKPALGVGAGNTPVIMDESCDVRLAVSSIIHSKTFDNGMICASEQSVIISDKIYEAAKKEFKDRGCHICSPEETQKLRETILINGALNAKIVGQSAHTIAKLAGFDVAEAAKILIGEVESVELEEQFAHEKLSPVLAMYKSKSFDDAVSKAARLVADGGYGHTSSIYINVGTGQEKIAKFSDAMKTCRILVNTPSSHGGIGDLYNFKLAPSLTLGCGSWGGNSVSENVGVKHLINIKTVAERRENMLWFRAPEKVYFKKGCLPVALAELKDVMNKKKVFIVTDAFLYKNGYTKCVTDQLDAMGIQHTTYYDVAPDPSLASATEGAEAMRLFEPDCIIALGGGSAMDAGKIMWVMYEHPEVNFLDLAMRFMDIRKRVYSFPKMGEKAYFIAVPTSSGTGSEVTPFAVITDERTGVKYPLADYELLPKMAIIDADMMMNQPKGLTSASGIDALTHALEAYASIMATDYTDGLALKAMKNIFAYLPSAYENGAADPVAREKMADASTLAGMAFANAFLGICHSMAHKLGAFHHLPHGVANALLINEVMRFNSVSIPTKMGTFSQYQYPHALDRYVECANFLGIAGKNDNEKFENLLKAIDELKEKVGIKKSIKEYGVDEKYFLDTLDAMVEQAFDDQCTGANPRYPLMKEIKEIYLKVYYGK

SEQ ID NO: 9 - protein, 1175 aa, Clostridium phytofermentans
MARKMKTMDGNTAAAHVSYAFTDVAAIYPITPSSPMADYTDMWATQGRKNIFGHEVLLSEMQSEAGAAGAVHGSLQAGALTTTYTASQGLLLMIPNMYKIAGELLPGVINVSARALASHALSIFGDHSDVYACRQSGFAMLCSGNVQETMDLGAVAHLTAIDGRVPFIHFFDGFRTSHEIQKISIWDYEDLKEMTNMEAVDAFRNRALNPEHPVQRGTAQNPDVFFQAREACNQYYDAIPELTQVYMDKVNAKIGTDYKLFNYYGAADAEHVVIAMGSVCDTIEETIDHMNASGAKVGLIKVRLYRPFSAKHLLETIPASVKQITVLDRTKEPGALGEPLYLDVVAALKDTQFHNLPVLTGRYGLGSKDTTPAQIIAVYNNKDKKNFTIGINDDVTHLSLDITENPDTANKGTTACKFWGLGADGTVGANKNSIKIIGDHTDKYAQAYFDYDSKKSGGVTISHLRFGDSPIKSTYLINKADFVACHMPAYVRRYNMVQDLKKGGTFLLNCSWNMEEIEKNLPGQVKRYMAQNNIKFYTIDGIQIGKEVGLGGRINTILQAAFFKLANIIPIEDAVKYMKDAATASYSKKGDDIVKMNHTAIDRGVDGLVEIKVPAEWANASDEDLAAKATVGRPEVLDYVNTILHKVNAQDGNSLPVSAFVDNADGTVPLGTAAYEKRGIAIDVPVWNPEICLQCNLCSYVCPHAVIRPVVMNEEQAANAPEGMKMVTMKQVEGKKFAITISVLDCTGCGSCAHVCPEVKGNKALSMDLLENHYDDQKYADYAASLETPVEILEKFKETTVKGSQFKQPLLEFSGACAGCGETPYAKLVTQLYGDRMYIANATGCSSIWGGSSPSTPYTVNKEGKGPAWANSLFEDNAEFGFGMQLAQTALRKRLIDSTENLVANSSSADVKAAAEEFLATQNNSTANAPATKNLLAALEACGCDNADRENILKNKSFLAKKSQWIFGGDGWAYDIGFGGLDHVIASGQDVNIMVFDTEVYSNTGGQSSKATPTGAIAQFAAAGKEVKKKDLAQIAMSYGYVYVAQIAQGADYNQCIKAITEAENYPGPSLIIAYAPCINHGIKGGMTGAQTEEKRAVEAGYWHLFRFNPTLKEEGKNPFVLDSKAPKASYQEFLQSEVRYNRLSRTNPERAAELFAKAEKDAKEKYEKLVKMAE

SEQ ID NO: 10 - DNA, 1320 bp, Clostridium phytofermentans
atggcagcaggaacattcaaaggcggcattcatccttatgaaggaaaagagctaacgaaggataaaccaaccactttattgctaccaaaaggagatcttgtgtatccaatgtctcaacacattggtaatccagcaaaacctattgttgcaaaaggcgacaaagttttagtaggtcaaaaaattggtgaagcagatggagtagtttccgcctgcatcattagctctgtatctggtacagtaaaagctgttgaaccaagattaaatgtggcaggcactatggtggaatccattgttgtggaaaatgataacgcttatactcaggtagaaggattcggagtagagagagattacgagactcttaaaaaggaacaaattcgttctattattaaggaagctggtattgtaggtatgggaggtgctggtttcccaacacacatcaagctaaccccaaaggatgatagcgcgattgattatttaatcattaatggttctgagtgtgaaccttatctaactagtgattatcgcatgatgttagaagagacaaatcgcttaattaaaggtattaagattacacttcgtttatttgaaaatgcaaaggctattattgcagtagaggataacaaaccagaagcaattagtatgcttacacatgcattaagaaatgagaacagaattgaattaaaagttattaaaacaaaatatcctcaaggtgcggaacgtgtgctaatttatgcaataacgggacgcaaaatgaattctactatgctaccatcggatattggatgtatcgtaaataatgtagatacgatgatttcagtttgtagagcagtagcagagaatacacctcttattaaaagagtcgtaacagtatctggagatgctgtgaaaaatcaagggaactttatcgtattaactggtactaattatagtgaactcgtagaagctgtaggaggatttagtgcaaaacctgcgaagctgatttctggtggacctatgatgggacttgctctttactccttagatataccagttacgaagacctctagtgcactattagcatttgcttcagatgaagtagcggatatggaggagggaccatgtatccgttgtggacgttgtgtggaagtttgcccaggtagaattgttccacagaaattaatggagtttgcagagcgttttgatgataaaggctttgaagggttaaatggtatggaatgttgtgaatgtggctgttgttcttatatctgtccagcaggacgtcatttaacacaggcttttaagcagtctaagagaagtattcttaacgaacgcaagaagtaa

SEQ ID NO: 11 - DNA, 1011 bp, Clostridium phytofermentans
ttgaaagatatgtataatgtctctgcatcaccgcacgtgcgtagtggtgtaacgacagctcagattatgagagatgttgcaattgcgttaatgcctgcttgtttatttggtatttatcaattcggtttctcagcatttttggtattattagtttcggtgacatcctgtgtggtatccgagtttttgtatgaaagattaatgaaacacccatatcgtccttatgagtgtagtgctctagttaccggtctattaatcggtatgaatatgcctgctaccattccagtatggattccaatggttggtggtgtatttgcaattatcgtagtaaaacagttatatggtggacttggacaaaactttatgaatcctgctcttgcagctagatgtttcttatccatctgttttacttctcgtatgacaacatttgcagtagatgcatttacaaattcaggtacttcaagtagaacattatatttatttaactatggttatgctggattagatggcgtcagcggtgcaactccacttgcagcgatgaaagcttccgaggcagctccaagtttacttgatatgttctttggtttccatggtggtgtgattggtgaaacaagtgccatgatgcttttaatcggtgcatgctatttattataccgtagaattatttccttacgaattccattgacatatatcgcaacatttgcagtatttataattttatttagcggcaaaggatttgatgtagaatatgtgttagctcagattcttggtggtggattaatattaggtgctttctttatggcaactgattacgtgacctgtccaattacgaagtatggtcaaatcctctttggtgtttgtcttggcgcgttaaccggattattccgtgtatttggtggttccgcagagggtgtatcttacgctattatcttctgtaacttattggtgccgttgattgaaaaaatcacgatgccaaggggcttcggaatgggaggtaagaaacttgcaaaataa

SEQ ID NO: 12 - DNA, 657 bp, Clostridium phytofermentans
ttgcaaaataagaaaaagtcaacaataattaaagatgcgattgcattatttgcgattaccttagtagcggctgttgcacttggttttgtatatgaaattacgaaagacccaatcgcagaagcagaagcaaaagcgaaggctaaagcatattcgatggtttttgccgatgcaaaattggtagatgataagaatgaagatgtgaatgccaaagtagattcttccaaagaatttttaacttctcaaggatttacttcaagtactatcaacgaagtatgtattgcaaaggatgaagccggaaatgcacttggctttgttatgactttaacttcttcagcaggatatggcggggatattaagtttacaatgggtgtaaaagcagacggaactttaacttcaatagaaattattagtatgaatgagacttcgggccttggtgcaaaagccaatgacgatagttttaaaggacaatattccgataaaaatgtagactcctttaaagttattaagtcagctgagagtaagactggtgatgatcaaattaatgccatcagtggtgcaacaatcacaagttctgcagtaacaggtacagtgaatgcaggtcttgcctttgcgaatgatttattagagaatggtgtaggaggtgttactcatgagtaa

SEQ ID NO: 13 - DNA, 762 bp, Clostridium phytofermentans
atgagtaaagcgttagagcgtatttataacggtgtaattaaagaaaatcctacatttgtcttaatgcttggtatgtgtccgactcttgcggttacaacttcagcaatcaatggtgtaggtatgggacttacgacaacagcagttcttatcatgtcaaacatgctaatttctatgcttcgtaaggctatccctgataaggtaagaatgccagcatttatcgtagtggtagcttccttcgtaactattgtgcagttattattgcaggcatatcttccttcattaaatgattcccttggtatctacatcccattgatcgttgttaactgtattatcctaggtagagcagaggcttatgcatcaaagtatccagtatacccatctatctttgatggtgtaggtatgggacttggatttaccgttggtttaactttaattggtttattccgtgaaatacttggtgcaggtactgcgtttggtttttctattatgccagatagctatgaaccattttctatcttcgtattagcaccgggtgcattctttgtccttgcgatgttgacagcccttcaaaataagttgaagttaaaatctgcaacaaatgttccaatggctgacaagcttgcatgtggcggtaactgcagcagttgtagcggtagtgcatgccatagcaatcatgagctacttgattccgtaaaagaagaagcaactaaaaaagcagcagctgaaaaggcacgtgcagctaatcagacagagaagaaatag

SEQ ID NO: 14 - DNA, 576 bp, Clostridium phytofermentans
atgaaggaattattactagtgcttattgcagcagcgctcgtgaataacgtagttttaagtcgtttcctcggcttatgtccgtttctcggcgtttctaaaaaaattagtacagcagcaggtatgggtggagcagtaatcttcgttattaccatagcctctgcattatgtagtgtaatctatgatgtggttttggttccacttgacttaaaatatatgaatacgattgtatttattattttaattgcagccttagttcagtttattgaaatgttcttaaagaagttctcaccaggtctatacaatgcactcggtgtataccttccattaatcacaacaaactgtgcagttctcggtgttgccatcgataacgtccaaaagggaaatggctttgtaattagtgttgtttatggtgctggtacggctattggtttcttaattgctattgttattatggcaggtgtaagagagcgaattgagaataacaatgtcacgaaatccttccaaggttcaccaattgtgttgattacagcaggattgatgtcaattgcctttatgggatttgcaggcttgttatag

SEQ ID NO: 15 - DNA, 849 bp, Clostridium phytofermentans
atgacgaatttagcattatttgacctcttatctaatactggtgtacttgctttcaatatgcaagggcttattacagcagcagctattgttggtggtgttggcttaatcattggtattcttcttggacttgcagccaaggtatttgaggttgaagtagatgaacgtgagttaatagtaagagatttattacctggtaataactgtggtggctgtggatatccaggttgtgatgggctagcaaaagcgattgcagctggtgaagcacctgtgagtggatgtcctgttgcaagcgccgaaattcacgctaaaattggtgaagttatgggtacagaagcaatagagagtgaacgtaatgttgcatttgtaaaatgtaatggtacctgtgataagacaaacgtaaagtatcactatactggaactccagattgtaagaagatttctacggtacctggaaatggcgagaagacttgtatctatggttgtatgggttatggtagctgtgtacgtgcttgtgcatttgatgcaattcatgttgtaaatggtattgcggtagtagataaagaaaaatgtgttgcatgtggaaaatgtattacagcgtgtccaaacgacttaattgaatttgttccagtaagttcaacttgcaaggtacaatgtaactctaaggataagggcaaagatgtgaacgctgcatgtagcgttggatgtattggatgtatgatgtgtgtgaaggtatgcgaaagcgatgcagtcaccgtaaccaataatcttgctcacattgattactctaagtgtactcattgcggtaagtgcgctgaaaagtgtccaagaaagattattaccattgcataa

SEQ ID NO: 16 - DNA, 1191 bp, Clostridium phytofermentans
atggcacgttttacactaccaagagatttatatcatggaaagggttctcttgcggaactaaaaaatttaacaggtaaaaaagcaattatcgttgttggaggcggctccatgaaacgttttggatttttggatagagccattgattacataaaagaagctggtatggaagtctctttgtttgaaaatgtagagccagaccctagtgtagaaactgtaatgaagggtgctgctgcgatgagagaattcgagccggattggattatatccatgggtggcggttctccaattgatgcagcaaaagcaatgtgggcattctatgaatatccagacacaacattcgaagatttgattgttccatttaacttcccaaccctacgtacaaaagcaaaattctgtgctatcccatctacctctggaacagcaactgaagtgactgcttttagcgtaattacagactatcacaagggtattaaatatcctctggcagactttaatattacaccagatgttgcaatcgtagatcctgatttagcagagacaatgcctgcaaaactcaccgcacatactggcatggatgctatgacacacgctgtggaagcatatgtttccacactacattgcgattataccgatcctcttgcaatgcatgctatccgtatggttcatgaatatttaaagtcttcttatgatggcaatatggatgcacgtgataagatgcacaatgcacaatgtttagctggtatggcattctccaacgcattacttggtattgttcactccatggctcataaaaccggcgctgcctactcaggaggtcatattgttcatggttgtgcaaatgcaatgtatctaccaaaagttattaaatttaattctaaaaatgaagatgcagcgaaacgttacgctgaaatcgcaactgcacttttcttaaaaggcaatacgactacagaacttgtagatgctctaattgaagaattaaatcagatgaaccgctccttgaatattccaagctgtatcaaggaatatgaaaatggtatcatcgatgaaaaagaattcttagaaaaattacctgaagtcgctgcaaatgctatctctgatgcttgtactggatcaaatccaagaatcccaacacaagaagagatggagaagttattaaaagcatgcttctataacgaagagattactttctaa

SEQ ID NO: 17 - DNA, 2619 bp, Clostridium phytofermentans
atgacgaaaaaagtggaattacagacaactggattagtagactctctcgaagcattaacagcaaaatttagagagttaaaagaagcacaagagctctttgctacctacactcaagagcaagtagataaaatcttctttgctgctgccatggctgccaatcagcaacgtattccgttagcaaagatggctgtagaagaaacgggtatgggtattgtagaagataaagtaattaagaatcattatgctgcagagtatatttacaatgcatacaaagatacaaaaacatgtggagtggttgaagaagatcctagcttcggtatcaaaaaaattgcagagccaatcggcgtagttgcagctgtaatcccaactaccaatcctacctccactgctatctttaaaacattactttgtttaaagactcgtaacgcaatcatcatcagcccacatcctcgtgctaagaactgtaccatcgcagctgctaaggtagttttagatgctgcagttgctgcaggtgctcctgctggtataattggatggattgatgttccatcacttgaattaaccaatgaagttatgaaaaatgcagacatcatccttgcaactggtggacctggtatggtaaaggctgcttattcttctggtaaaccagcacttggtgttggcgcaggtaatacccctgttattatggatgaaagctgcgatgttcgccttgcagtaagctctattattcactctaagacatttgataacggtatgatttgtgcttccgagcaatccgtaattattagtgataagatttatgaagctgctaagaaagaattcaaggatcgtggttgccacatctgctccccagaagagactcagaagcttcgtgaaacaatcctaattaatggtgctcttaacgctaaaattgttggacaaagcgctcatacgattgcaaagcttgcaggatttgatgtagcagaagctgctaagattttaattggtgaagtagaatccgttgaactagaagaacaatttgcacacgagaaactttctccagttcttgctatgtacaaatcaaaatcctttgatgatgcagtaagcaaagctgctcgtcttgttgcagatggcggttatggccatacttcttccatctatattaatgtaggtaccggacaagaaaagattgcaaagttttctgatgctatgaagacttgccgtattcttgtaaatacaccatcctcccatggtggtatcggtgacctttataactttaaattagctccatctcttactcttggttgtggctcctggggcggtaactctgtatcagaaaacgtaggagtaaagcacttaatcaacattaagacagttgctgagaggagagaaaacatgctttggtttagagcacctgagaaagtatactttaagaagggttgtttaccagtagccctcgcagaattaaaagatgtaatgaataaaaagaaagtattcattgtaaccgatgctttcctttataaaaatggctatacaaaatgtgttactgatcagttagatgctatgggaattcagcatactacttactatgatgttgctccagatccatctttagctagtgctacagaaggtgcagaagcgatgagactcttcgagccagactgtattatcgcactcggtggtggttctgcaatggatgccggaaagattatgtgggttatgtatgaacaccctgaagtaaacttccttgaccttgcaatgcgtttcatggatattagaaagcgtgtttactccttccctaagatgggcgaaaaagcttactttatcgcagttccaacttcctccggtactggttctgaagttacaccatttgctgttattaccgatgagagaactggcgtaaaatatccacttgcagattacgaattacttcctaagatggctattattgatgccgatatgatgatgaatcaacctaagggattaacttctgcttccggtattgatgcccttacccatgcattagaggcatatgcttctatcatggctactgactatacggatggtttagcattaaaagctatgaagaatatcttcgcttaccttccaagcgcatatgaaaatggtgccgctgatccggttgcaagagaaaagatggcagatgcttctaccttagctggtatggcattcgcaaatgcattcttaggaatttgccactccatggctcataaattaggtgcattccaccacttaccacacggtgtagcaaacgcactcttaatcaacgaagtaatgcgctttaactccgttagcattcctacaaagatgggtactttctctcaataccaatacccacatgcgttagatcgttatgtagaatgtgcgaacttcttaggtattgccggaaagaacgacaatgagaaattcgaaaaccttcttaaggcaattgatgaattaaaagaaaaagttggtatcaagaaatccatcaaagaatatggcgtagacgagaaatatttcttagatactttagatgctatggttgaacaggctttcgatgatcagtgtactggtgctaacccaagatatccattaatgaaggaaatcaaggaaatctatcttaaagtgtactacggtaaataa

SEQ ID NO: 18 - DNA, 3528 bp, Clostridium phytofermentans
atggctagaaaaatgaaaaccatggatggtaataccgctgcggcacacgtgtcatatgcatttaccgatgtagcggcaatctatccaatcacaccatcttcaccaatggctgactacacagatatgtgggcaactcagggaagaaagaacatcttcggacacgaagtattattatccgagatgcaatctgaagcaggtgcagcaggtgctgttcacggttctttacaggcaggtgcattaactacaacctacaccgcgtcccaaggtttattattaatgatccctaatatgtataagatcgctggtgagttattaccaggcgttattaatgtttctgcacgtgctcttgcaagtcatgcactttccatctttggcgatcattccgacgtttacgcttgtcgtcaatcaggatttgctatgctttgctccggtaatgttcaggaaactatggacttaggtgctgttgctcacttaacagctatcgacggtcgtgttccatttatccatttctttgatggatttagaacatctcatgaaattcaaaaaatctctatctgggattacgaagatttaaaagaaatgactaatatggaagctgtagatgcattccgtaatagagctttaaatccagaacacccagttcaaagaggtactgctcagaaccctgacgtattcttccaggcaagagaagcttgtaaccaatactatgatgcaattcctgaacttactcaagtttacatggacaaggttaacgctaaaatcggtactgactataaattattcaactactacggtgctgctgatgcagagcatgttgtcattgctatgggttcagtttgcgatactatcgaagagacaatcgaccatatgaatgcaagtggtgctaaggttggtcttatcaaagttcgtctttacagaccattctccgctaagcatttattagagactattcctgcatctgttaagcagattactgttcttgatagaacaaaagagccaggtgctcttggtgagcctttatacttagacgttgtagctgctcttaaggatacacaattccataatcttcctgtattaacaggccgctatggtttaggttccaaagatactacaccagctcagattatcgctgtttacaacaacaaggataagaagaatttcacaatcggtatcaacgatgatgtaactcatctttctcttgatatcacagagaatccagatacagctaacaagggtacaacagcttgtaagttctggggacttggtgctgatggtactgtaggtgctaataagaactccatcaagattatcggtgaccatacagataagtacgctcaggcttactttgattatgactccaagaaatccggtggtgttactatctcccacttacgtttcggtgatagcccaatcaaatccacttacttaatcaacaaagctgacttcgttgcatgtcacatgccagcttacgttagaagatataacatggtacaggatcttaagaagggtggtacattcctccttaactgttcttggaacatggaagaaatcgagaagaaccttcctggtcaggtaaaacgttatatggctcagaacaacattaagttctacaccatcgacggtatccagattggtaaagaagttggtcttggtggacgtattaatactatccttcaggctgctttcttcaaattagctaacatcattcctattgaggatgctgtaaaatatatgaaagatgctgctactgcttcttattctaagaagggtgatgacatcgttaagatgaaccataccgcaattgaccgtggtgttgatggtctcgttgaaattaaagttcctgctgaatgggctaacgcttccgacgaggacttagctgctaaagcaactgttggtagaccagaagttcttgattatgttaacacaattcttcacaaggtaaatgctcaggacggtaacagtcttccagtttctgctttcgttgacaatgcagatggtactgtacctctaggaacagctgcatacgagaaacgtggtattgcaatcgacgttccagtatggaatccagaaatttgtttacagtgtaacctttgttcttacgtatgtccacatgcagtaatccgtccagttgttatgaacgaagaacaagctgctaatgctccagaaggcatgaagatggttactatgaagcaagtagaaggcaagaagtttgctatcactatctccgtacttgactgtacaggttgtggaagctgtgctcatgtttgtccagaagttaagggtaataaggctcttagcatggatttacttgagaaccactacgatgatcagaagtatgctgattacgctgcatccttagaaactcctgttgaaatccttgagaaattcaaagagacaactgttaagggtagccagttcaaacagccattacttgagttctccggagcttgtgctggttgtggtgaaacaccttacgctaaattagttactcagttatatggtgatagaatgtatattgcaaacgctactggatgttcttctatctggggtggttcttctccttctacaccttatacagttaataaagaaggcaagggtccagcttgggctaactccttattcgaggataatgctgaattcggtttcggtatgcaattagctcaaacagctcttagaaaacgccttatcgattctacagagaatttagtagctaattcatcaagtgctgatgttaaggctgctgctgaagagttccttgcaacacagaataactccactgcaaatgctcctgctactaagaatttactcgctgcattagaagcttgcggatgtgacaatgcagatagagaaaacatcttaaagaacaagagcttcttagctaagaagtctcaatggatctttggtggtgacggttgggcttacgatatcggtttcggcggtcttgaccacgtaatcgcttccggccaggatgtaaacatcatggtattcgatactgaagtttactccaatacaggtggacagtcctctaaggctacaccaacaggtgctatcgctcagttcgctgctgctggtaaagaagttaagaagaaagaccttgctcaaattgctatgagctatggctacgtatatgtagcacagatcgctcagggtgctgattacaatcagtgtatcaaggctatcacagaagctgagaactatccaggtccatccttaattatcgcttatgctccatgtatcaaccatggtatcaagggcggtatgacaggtgctcagacagaagagaaacgtgctgttgaagctggttactggcacttattcagattcaatcctactttaaaagaagaaggaaagaatccattcgtgttagattctaaggctccaaaggctagctaccaagaattccttcagagcgaggttcgttacaacagacttagcagaacaaatccagaaagagctgcagaattatttgcaaaggctgagaaggatgctaaggagaaatacgagaagcttgtaaagatggctgagtaa